(19) United States
(12) Patent Application Publication
(10) Pub. No.: US 2019/0175049 A1
(43) Pub. Date: Jun. 13, 2019

(54) TECHNIQUES FOR ANALYZING NON-VERBAL MARKERS OF CONDITIONS USING ELECTROPHYSIOLOGICAL DATA

(71) Applicant: Massachusetts Institute of Technology, Cambridge, MA (US)

(72) Inventor: Caroline Welling, Hanover, NH (US)

(21) Appl. No.: 16/206,639

(22) Filed: Nov. 30, 2018

Related U.S. Application Data
(60) Provisional application No. 62/593,535, filed on Dec. 1, 2017.

(51) Int. Cl. A61B 5/0484; A61B 5/00; G06N 20/10; A61B 5/0478

(52) U.S. Cl. CPC A61B 5/04842; A61B 5/7289; A61B 5/0478; A61B 5/7225; G06N 20/10

(57) ABSTRACT

Embodiments related to analyzing brain activity of a subject to identify signs associated with binocular rivalry. Sensed electrical activity of a subject's brain is received over a time period while the subject is exposed to a visual stimulus. The sensed electrical activity comprises a first frequency band associated with a first frequency of a first image presented to the subject's left eye, a second frequency band associated with a second frequency of a second image presented to the subject's right eye. A set of events in the time period is determined based on the frequency bands, wherein an event is associated with a change from a previous perceptual event to a new perceptual event. A metric for the subject is determined based on the set of events. The metric is analyzed to determine whether the subject exhibits signs associated with a condition that is associated with binocular rivalry.

### RELATED APPLICATIONS

This Application claims priority under 35 U.S.C. § 119(e) to U.S. Provisional Application, Ser. No. 62/593,535, entitled “TECHNIQUES FOR ANALYZING NON-VERBAL MARKERS OF CONDITIONS USING ELECTROPHYSIOLOGICAL DATA,” filed Dec. 1, 2017, which is herein incorporated by reference in its entirety.

### TECHNICAL FIELD

Embodiments of the present invention relate to techniques for analyzing non-verbal markers of conditions using electrophysiological data.

### BACKGROUND

It is often desirable to test subjects for various conditions, such as Austism Spectrum Disorder, fragile X syndrome (or associated symptoms), Asperger syndrome, and/or other conditions. Current behavioral assays for these conditions and/or associated symptoms often involve administering a stimulus to the subject, and requiring the subject to provide physical or verbal responses (e.g., audible responses, physical movements, etc.). However, such tests can be difficult to perform on certain subjects, such as young subjects and/or inaudible subjects, such as non-verbal human subjects as well as animal subjects. This can result in certain groups and/or group members being ineligible for behavioral assays, and/or can also makes it difficult or impossible to test the efficacy of drug treatments directed to these conditions. Further, such tests are often subject to human error, which can be compounded when testing subjects with various physical and mental conditions.

### SUMMARY OF THE INTENTION

The present disclosure describes methods and apparatus to evaluate subjects suffering from conditions that are associated with binocular rivalry (e.g., an Autism Spectrum Disorder, or Fragile X syndrome). Various aspects enable analysis of groups that cannot be achieved with conventional approaches including, for example, non-verbal subjects (e.g., non-verbal human subjects or animal subjects).

Some embodiments relate to a computerized method for analyzing brain activity of a subject associated with the subject's vision to identify signs associated with binocular rivalry. The method includes receiving sensed electrical activity of a subject's brain over a time period while the subject is exposed to a visual stimulus, wherein the sensed electrical activity includes a first frequency band associated with a first frequency of a first image presented to the subject's left eye, and a second frequency band associated with a second frequency of a second image presented to the subject's right eye. The method includes determining, based on the first and second frequency bands, a set of events in the time period, wherein an event in the set of events is associated with a change from a previous perceptual event triggered by observation of the first and second images by the subject to a new perceptual event triggered by observation of the first and second images by the subject. The method includes determining, based on the set of events, a metric for the subject. The method includes analyzing the metric to determine whether the subject exhibits signs associated with a condition that is associated with binocular rivalry.

In some examples, determining the metric includes determining an average perceptual event rate, determining a depth of perceptual suppression based on a ratio of dominant to mixed percepts, or both.

In some examples, the first frequency band includes a power of the first frequency band over the time period, the second frequency band includes a power of the second frequency band over the time period, and determining the set of events includes analyzing the data of the first frequency band and the second frequency band using a Fourier transform analysis to determine the average perceptual event rate for the subject.

In some examples, the computerized method further includes determining the previous perceptual event and the new perceptual event, wherein the previous perceptual event is associated with the user perceiving just the first image, the user perceiving just the second image, or the user perceiving a merged image of the first image and the second image, and the new perceptual event is associated with the user perceiving just the first image, the user perceiving just the second image, or the user perceiving a merged image of the first image and the second image.

In some examples, receiving the sensed electrical activity includes receiving EEG data, data sensed using intracranial electrodes, or some combination thereof.

In some examples, analyzing the metric includes training a model to determine whether the metric is indicative of a condition that is associated with binocular rivalry, comprising training the model using training data in which timing information associated with the visual stimulus is known, and classifying the metric based on the model to determine whether the metric is indicative of a condition that is associated with binocular rivalry.

In some examples, the model is a support vector machine classifier.

Some embodiments relate to a computerized apparatus for analyzing brain activity of a subject associated with the subject's vision to identify signs associated with binocular rivalry, the system comprising a processor in communication with a memory storing instructions that, when executed by the processor, cause the processor to receive sensed electrical activity of a subject's brain over a time period while the subject is exposed to a visual stimulus, wherein the sensed electrical activity includes a first frequency band associated with a first frequency of a first image presented to the subject's left eye, and a second frequency band associated with a second frequency of a second image presented to the subject's right eye. The instructions cause the processor to determine, based on the first and second frequency bands, a set of events in the time period, wherein an event in the set of events is associated with a change from a previous perceptual event triggered by observation of the first and second images by the subject to a new perceptual event triggered by observation of the first and second images by the subject. The instructions cause the processor to determine, based on the set of events, a metric for the subject. The instructions further cause the processor to analyze the metric to determine whether the subject exhibits signs associated with a condition that is associated with binocular rivalry.

In some examples, determining the metric includes determining an average perceptual event rate, determining a depth of perceptual suppression based on a ratio of dominant to mixed percepts, or both.

In some examples, the first frequency band includes a power of the first frequency band over the time period, the second frequency band includes a power of the second frequency band over the time period, and determining the set of events includes analyzing the data of the first frequency band and the second frequency band using a Fourier transform analysis to determine the average perceptual event rate for the subject.

In some examples, the instructions further cause the processor to determine the previous perceptual event and the new perceptual event, wherein the previous perceptual event is associated with the user perceiving just the first image, the user perceiving just the second image, or the user perceiving a merged image of the first image and the second image, and the new perceptual event is associated with the user perceiving just the first image, the user perceiving just the second image, or the user perceiving a merged image of the first image and the second image.

In some examples, receiving the sensed electrical activity includes receiving EEG data, data sensed using intracranial electrodes, or some combination thereof.

In some examples, analyzing the metric includes training a model to determine whether the metric is indicative of a condition that is associated with binocular rivalry, wherein the model is trained using training data in which timing information associated with the visual stimulus is known, and classifying the metric based on the model to determine whether the metric is indicative of a condition that is associated with binocular rivalry.

In some examples, the model is a support vector machine classifier.

Aspects of this disclosure provide a method of diagnosing or prognosing someone with a condition that is associated with binocular rivalry. In some aspects, provided herein is a method of evaluating a subject using anyone of the computerized methods disclosed herein. In some embodiments, a method of evaluating a subject comprises providing a visual stimulus to the subject, and evaluating brain activity of the subject according to any one of the computerized methods described herein to determine a metric. In some embodiments, a subject is likely to have a condition that is associated with binocular rivalry if the metric is less than a reference metric. In some embodiments, a metric is average perceptual event rate. In some embodiments, a metric is depth of perceptual suppression based on a ratio of dominant to mixed percepts, or both.

In some embodiments, a visual stimulus comprises presenting to the subject a first frequency-tagged image to the left eye and a second frequency-tagged image to the right eye, wherein the frequency of the first frequency-tagged image is different from the frequency of the second frequency-tagged image. In some embodiments, the first image is presented exclusively to the left eye and second image is presented exclusively to the right eye. In some embodiments, the first image is presented exclusively to the left eye and second image is presented exclusively to the right eye using a stereoscope or lens. In some embodiments, the images are displayed on a digital monitor or headset. In some embodiments, the first and second images on a digital monitor through a stereoscope, wherein the stereoscope reflects the left and right sides of the monitor into the subject's left and right eyes.

In some embodiments of a method of evaluating a subject as disclosed herein, the condition that is associated with binocular rivalry is an Autism Spectrum Disorder (ASD) or Fragile X syndrome. In some embodiments, ASD is one or more condition in the following group: Autistic Disorder, Asperger's Disorder, Rett's Disorder, Childhood Disintegrative Disorder, and Pervasive Developmental Disorder Not Otherwise Specified (PDD-NOS).

In some embodiments, a method of evaluating a subject further comprises clinically evaluating the subject to determine if the subject has an ASD. In some embodiments, clinically evaluating the subject is guided by the diagnostic criteria set forth in the Diagnostic and Statistical Manual of Mental Disorders (DSM, e.g., DSM-IV, DSM-V or DSM-IV-TR).

In some embodiments, a subject is evaluated over time. Accordingly, is some embodiments, a method of evaluating a subject further comprising determining a metric in the subject at at least one additional time point (e.g., multiple times over the course of a week, a month, a year, 2 years 5 years, 10 years, 20 years, or 50 years, or 80 years or over a lifetime of a subject).

In some embodiments, a subject has been previously diagnosed to have a mental disorder. In some embodiments, a mental disorder is an ASD, Fragile X syndrome, an anxiety disorder, attention deficit hyperactivity disorder (ADHD), a sensory processing disorder, or Social (Pragmatic) Communication Disorder.

In some embodiments, a subject is a child and shows delays in developing language skills. In some embodiments, the ASD that subject is previously diagnosed with is one or more condition in the following group: Autistic Disorder, Asperger's Disorder, Rett's Disorder, Childhood Disintegrative Disorder, and PDD-NOS. In some embodiments, a subject is human. In some embodiments, a human is a child. In some embodiments, a subject is an infant. In some embodiments, a subject is a lab animal. In some embodiments, a lab animal is a rodent, a non-human primate or a bird. In some embodiments, a rodent is a mouse, a rat, or a ferret, and the non-human primate is a macaque or a marmoset. In some embodiments, a subject is non-verbal. In some embodiments, a subject is high-functioning.

In some embodiments, a reference metric is an average of metric values of all subjects in a reference population. In some embodiments, a reference population consists of individuals that do not suffer from a condition that is associated with binocular rivalry. In some embodiments, the condition that is associated with binocular rivalry is an ASD. In some embodiments, reference population consists of individuals that do not suffer from any mental disorder.

In some embodiments, determining the average perceptual event rate comprises analyzing the detected brain activity using a fast Fourier transform (FFT) analysis to determine the average perceptual event rate.

The computerized methods provided herein may be used to measure the efficacy of a treatment of a condition that is associated with binocular rivalry. Accordingly in some aspects, provided herein is a method of determining the efficacy of a treatment in a subject suffering from a condition that is associated with binocular rivalry. In some embodiments, a method of determining the efficacy of a treatment comprises providing a visual stimulus to the subject, and detecting brain activity of the subject by any one of the computerized methods as provided herein to determine a metric. In some embodiments, wherein the treatment is said to be efficacious if the metric is increased after the administration of the treatment.

In some embodiments, a metric is average perceptual event rate. In some embodiments, a metric is depth of perceptual suppression based on a ratio of dominant to mixed percepts, or both.

In some embodiments, a visual stimulus comprises presenting to the subject a first frequency-tagged image to the left eye and a second frequency-tagged image to the right eye, wherein the frequency of the first frequency-tagged image is different from the frequency of the second frequency-tagged image. In some embodiments, the first image is presented exclusively to the left eye and second image is presented exclusively to the right eye. In some embodiments, the first image is presented exclusively to the left eye and second image is presented exclusively to the right eye using a stereoscope or lens. In some embodiments, the images are displayed on a digital monitor or headset. In some embodiments, the first and second images on a digital monitor through a stereoscope, wherein the stereoscope reflects the left and right sides of the monitor into the subject's left and right eyes.

In some embodiments, a treatment is a behavioral therapy or administration of a pharmaceutical agent. In some embodiments, a pharmaceutical agent is a selective serotonin reuptake inhibitor. In some embodiments, a selective serotonin reuptake inhibitor is citalopram, fluoxetine or sertraline. In some embodiments, a pharmaceutical agent is a gamma-Aminobutyric acid (GABA) receptor modulator. In some embodiments, a GABA receptor modulator is a GABA receptor agonist or a GABA receptor positive allosteric modulator. In some embodiments, a GABA receptor agonist is clobazam or arbaclofen. In some embodiments, a pharmaceutical agent affects irritability and aggression, aberrant social behavior, hyperactivity and inattention, repetitive behaviors, cognitive disorders, or insomnia. In some embodiments, a pharmaceutical agent affecting irritability and aggression is Risperidone, Aripiprazole, Clozapine, Haloperidol, or Sertraline. In some embodiments, a pharmaceutical agent affecting aberrant social behavior is Risperidone, Haloperidol, Oxytocin, or Secretin. In some embodiments, a pharmaceutical agent affecting hyperactivity and inattention is Methylphenidate or Venlafaxine. In some embodiments, a pharmaceutical agent affecting repetitive behaviors is Fluoxetine, Citalopram, or Bumetanide. In some embodiments, a pharmaceutical agent affecting cognitive disorders is Memantine or Rivastigmine. In some embodiments, a the pharmaceutical agent affecting insomnia is Mirtazapine or Melatonin. In some embodiments, a treatment is experimental.

In some embodiments, any one of the methods of determining the efficacy of a treatment is used to assay compounds to identify efficacious drugs.

In some embodiments of any one of the methods to determine the efficacy of a treatment for a condition associated with binocular rivalry, a condition that is associated with binocular rivalry is an ASD or Fragile X syndrome.

Methods provided herein may guide the treatment of a subject suffering from a condition associated with binocular rivalry. Accordingly, in some aspects, provided herein is a method of treating a subject diagnosed with a condition that is associated with binocular rivalry. In some embodiments, a method of treatment as disclosed herein comprises providing a visual stimulus to the subject, detecting brain activity of the subject according to the method of any one of the methods disclosed herein to determine a metric, and administering a treatment to the subject if the metric of the subject is less than a reference metric.

In some embodiments, a method of treating a subject diagnosed with a condition that is associated with binocular rivalry comprises directing or ordering a test on the subject to determine a metric according to any one of the method disclosed herein to determine a metric, and administering a treatment to the subject if the metric of the subject is less than a reference metric. In some embodiments, a method of treating a subject diagnosed with a condition that is associated with binocular rivalry comprises selecting the subject on the basis that the subject has a metric that is less than a reference metric, and administering a therapeutic treatment to the subject.

In some embodiments, a condition that is associated with binocular rivalry is an ASD or Fragile X syndrome. In some embodiments, an ASD is one or more condition in the following group: Autistic Disorder, Asperger's Disorder, Rett's Disorder, Childhood Disintegrative Disorder, and Pervasive Developmental Disorder Not Otherwise Specified (PDD-NOS).

In some embodiments, a metric is average perceptual event rate. In some embodiments, a metric is depth of perceptual suppression based on a ratio of dominant to mixed percepts, or both.

In some embodiments, a visual stimulus comprises presenting to the subject a first frequency-tagged image to the left eye and a second frequency-tagged image to the right eye, wherein the frequency of the first frequency-tagged image is different from the frequency of the second frequency-tagged image. In some embodiments, the first image is presented exclusively to the left eye and second image is presented exclusively to the right eye. In some embodiments, the first image is presented exclusively to the left eye and second image is presented exclusively to the right eye using a stereoscope or lens. In some embodiments, the images are displayed on a digital monitor or headset. In some embodiments, the first and second images on a digital monitor through a stereoscope, wherein the stereoscope reflects the left and right sides of the monitor into the subject's left and right eyes.

In some embodiments, a treatment comprises a behavioral treatment or administration of a pharmaceutical agent. In some embodiments, a pharmaceutical agent is a selective serotonin reuptake inhibitor. In some embodiments, a selective serotonin reuptake inhibitor is citalopram, fluoxetine or sertraline. In some embodiments, a pharmaceutical agent is a gamma-Aminobutyric acid (GABA) receptor modulator. In some embodiments, a GABA receptor modulator is a GABA receptor agonist or a GABA receptor positive allosteric modulator. In some embodiments, a GABA receptor agonist is clobazam or arbaclofen. In some embodiments, a pharmaceutical agent affects irritability and aggression, aberrant social behavior, hyperactivity and inattention, repetitive behaviors, cognitive disorders, or insomnia. In some embodiments, a treatment is experimental.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a diagram showing a visual stimulus used to invoke binocular rivalry in a subject, according to some examples;

FIG. 2 is a graph showing perceptual suppression between a control group of subjects and an autistic group of subjects, according to some examples;

FIG. 3 is a diagram showing an exemplary system for analyzing electrophysiological data of a subject, according to some embodiments;

FIG. 4 is a flow diagram showing a computerized method for analyzing electrophysiological data to determine whether a subject exhibits signs associated with a condition, according to some embodiments;

FIG. 5 is a graph showing an example of sensed frequency and power of EEG signals for a subject exposed to a visual stimulus, according to some embodiments;

FIG. 6 includes a smoothed and unsmoothed graph of EEG signals overlaid on physical response information from a subject, according to some embodiments;

FIG. 7 is a graph showing averaged data for five seconds before, and five second after, a reported perceptual switch, according to some embodiments;

FIG. 8 includes graphs of reported switches for a control group and an ASC group, according to some embodiments;

FIG. 9 is a flow diagram showing a computerized method for calculating a subject's oscillation frequency, according to some embodiments;

FIG. 10 includes graphs illustrating steps used to calculate a subject's oscillation frequency, according to some embodiments;

FIGS. 11A and 11B include graphs comparing the number of switches per trial with the oscillation frequency for the control group and the ASC group, according to some embodiments;

FIG. 12 shows bar graphs comparing the number of switches per trial in the first graph with the proportion of perceptual suppression in the second graph, according to some embodiments;

FIG. 13 includes graphs showing the accuracy of the analysis techniques in relation to the time of a subject's reported transition, according to some embodiments;

FIG. 14 is a diagram illustrating the reduced GABAergic action in the autistic brain, according to some examples;

FIG. 15 includes two graphs comparing the depth of perceptual suppression for a placebo and clobazam and bumetanide, respectively, according to some examples;

FIG. 16 is a table that illustrates the subjects used in an exemplary study to test non-verbal markers associated with binocular rivalry, according to some examples;

FIG. 17 illustrates the study protocol and associated visual stimulus used in the exemplary study, according to some examples;

FIG. 18 includes two graphs comparing the depth of perceptual suppression for a placebo to clobazam and a placebo to arbaclofen, according to some examples;

FIG. 19 shows a bar graph comparing the number of switches per trial for subjects treated with a placebo and arbaclofen, according to some examples; and

FIG. 20 shows bar graphs comparing the proportion of perceptual suppression in control group and ASC groups in the first graph with the number of switches per trial of the same groups in the second graph, according to some examples.

### DETAILED DESCRIPTION

Described herein are various embodiments for objective, non-verbal techniques to measure conditions that are associated with binocular rivalry, such as ASD and Fragile X syndrome. Applicant has recognized and appreciated that electrophysiological data can be used to obtain and analyze non-verbal markers indicative of conditions that are associated with binocular rivalry, such as perceptual switches (e.g., the number of perceptual switches in a time period) and/or the depth of perceptual suppression (e.g., the ratio of dominant to dominant plus mixed percept durations) when the subject is exposed to a vision stimulus. Such non-verbal markers can be used to determine whether the subject exhibits signs associated with various conditions (e.g., using machine learning techniques and/or other data analysis techniques).

A challenge of researching conditions (e.g., autism) is a lack of robust markers of the condition, whether behavioral or neural. This can limit research in one or more ways. For example, because current behavioral assays often limit test groups to studying high-functioning, verbal individuals, a large subset of individuals who lack language are not studied. As another example, there are few paradigms that can track autistic behavior throughout development. As a further example, there are no behavioral paradigms that afford a meaningful connection between animals and humans. Being able to draw a connection between animals and humans can be important, for example, for drug development because animals lack face validity and predictive validity.

It is therefore desirable to develop non-verbal techniques to measure markers associated with conditions. Since non-verbal reports do not rely on verbal and/or physical responses, such non-verbal techniques can allow for, e.g., development of animal models, development of diagnostic tools for non-verbal patients, tracking neural responses throughout development (e.g., rivalry), and/or a more direct measure of perception. Such techniques, as discussed further below, can leverage electrophysiological measurements to identify and analyze non-verbal condition markers to test subjects (including subjects that cannot otherwise be tested using behavioral assays). Various embodiments implement electrophysiological measurements to improve over conventional approaches for analyzing conditions associated with binocular rivalry, expand on potential subjects, and develop condition markers unavailable to conventional implementation.

As discussed further herein, Applicant has discovered a candidate biomarker (e.g., of Autism Spectrum Disorder (ASD) and/or other conditions) in visual perception: reduced perceptual suppression during binocular rivalry. The reduced perceptual suppression links to the reduced action of the inhibitory neurotransmitter γ-aminobutyric acid (GABA) in the autistic brain. Applicant has discovered that electrophysiological data can be used to analyze non-verbal markers to measure conditions associated with binocular rivalry, such as the number of switches per trial and/or the depth of perceptual suppression. This candidate biomarker is test-retest reliable, can be replicated, can be used to predict clinical measures (e.g., gold-standard clinical measures) of autism symptom severity in social cognition (e.g., to test Autism Diagnostic Observation Schedule (ADOS) scores), and can be used to index drug responses.

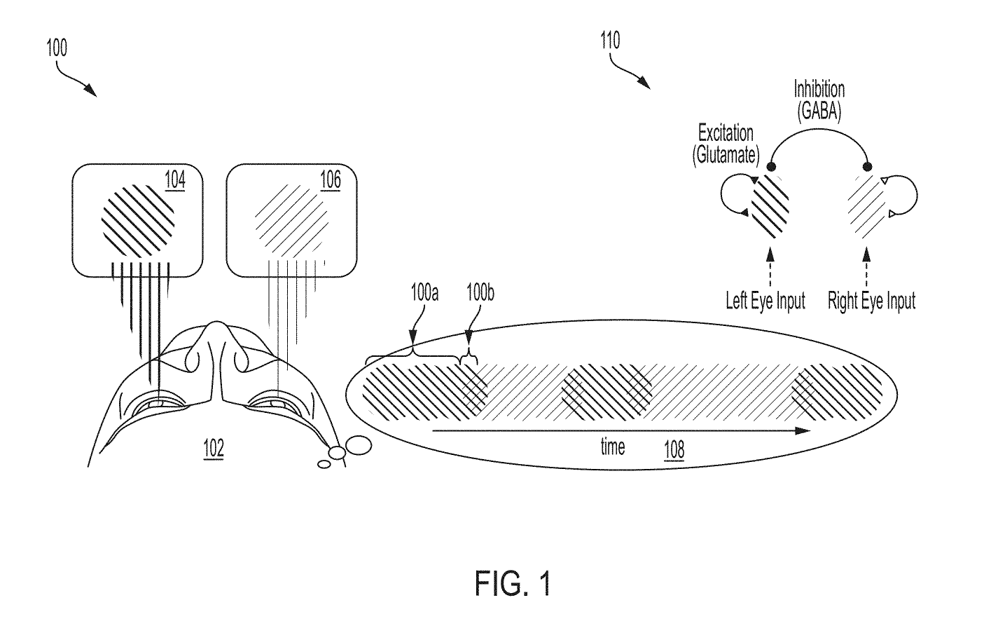

Binocular rivalry is a phenomenon of visual perception in which a subject's visual perception alternates between different images presented to each eye. A visual stimulus can be used to trigger binocular rivalry by presenting very different images to each eye: a first image is seen for a few moments, then the other second image, then the first, and so on. During transitions from perceiving one image to the other image, a composite of the two images may be seen. FIG. 1 is a diagram 100 showing a visual stimulus used to invoke binocular rivalry in a subject 102, according to some examples. The visual stimulus involves presenting a first image 104 (which has white diagonal stripes that extend downward from left-to-right) to the left eye and a second image 106 (which has white diagonal stripes that extend upward from left-to-right) to the right eye. The images can be colored differently, such as a red first image 104 and a blue second image 106. The images can be presented to the user at different rates (e.g., times per second), as well as at different times, as discussed further herein. As shown in the timeline 108, over time the subject first perceives the first image 104 (108a), then the subject perceives a mix of the first and second images 104 and 106 as the subject transitions to perceiving the second image 106 (108b), and so on.

As illustrated in the portion 110 of FIG. 1, binocular rivalry can relay the balance of inhibitory (neurotransmitter y-aminobutyric acid (GABA)) and excitatory (glutamate) dynamics in the subject's visual system, which can be used to diagnose whether the subject exhibits signs of a condition that is associated with binocular rivalry, as discussed further below.

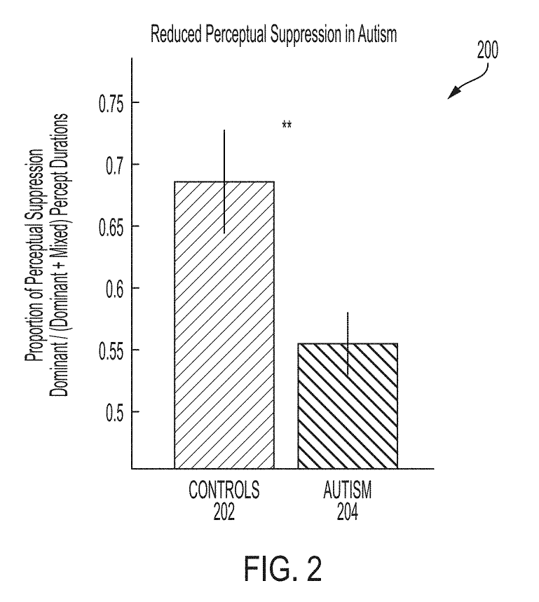

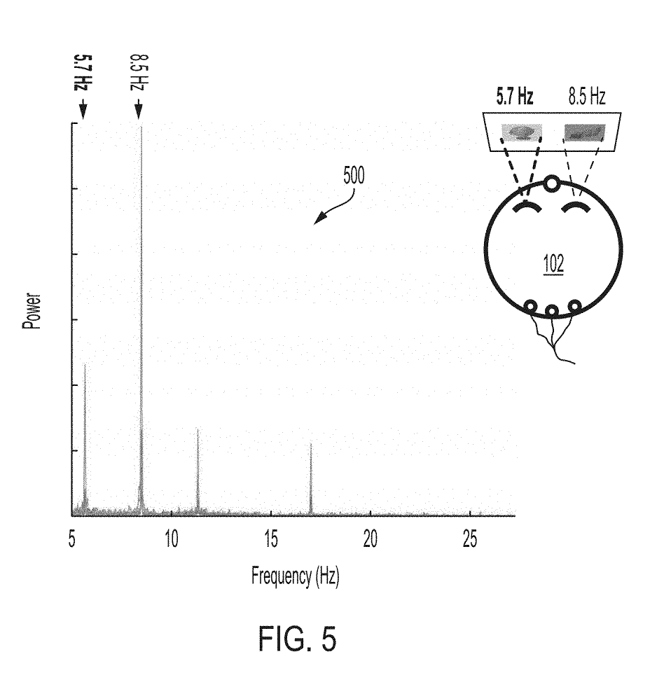

As noted above, electrophysiological data can be analyzed to determine a subject's perceptual suppression when observing a visual stimulus. FIG. 2 is a graph 200 showing perceptual suppression between a control group 202 of subjects and an autistic group 204 of subjects, according to some examples. FIG. 2 and other figures (e.g., FIGS. 5-8, 10-13 and 15) graphically depict exemplary data obtained during the first exemplary study discussed further below in the Examples section. Since these figures help illustrate concepts being discussed herein, the figures are presented in conjunction with other figures as non-limiting examples to help illustrate the techniques disclosed herein. As a general matter, the test involved two groups of subjects—a control group and an Autism Spectrum Condition (“ASC”) group, and the exemplary graphs illustrate the detection and analysis of non-verbal markers associated with binocular rivalry for ASC, as discussed further herein. A more detailed description of the first exemplary study is presented in the Examples section below. Additionally, while the abbreviation ASC is used herein, a person of skill in the art will appreciate that the formal definition is Autism Spectrum Disorder.

As illustrated in the graph, individuals with autism exhibit atypical binocular rivalry, including slower binocular rivalry oscillations (e.g., perceptual switches between the two images), which are marked by reduced perceptual suppression. The graph 200 shows that the control group 202 exhibits a proportion of perceptual suppression (e.g., the proportion of dominant perceptions divided by the sum of dominant and mixed perceptions) of approximately 0.69, while the autistic group exhibits a proportion of perceptual suppression of approximately 0.55.

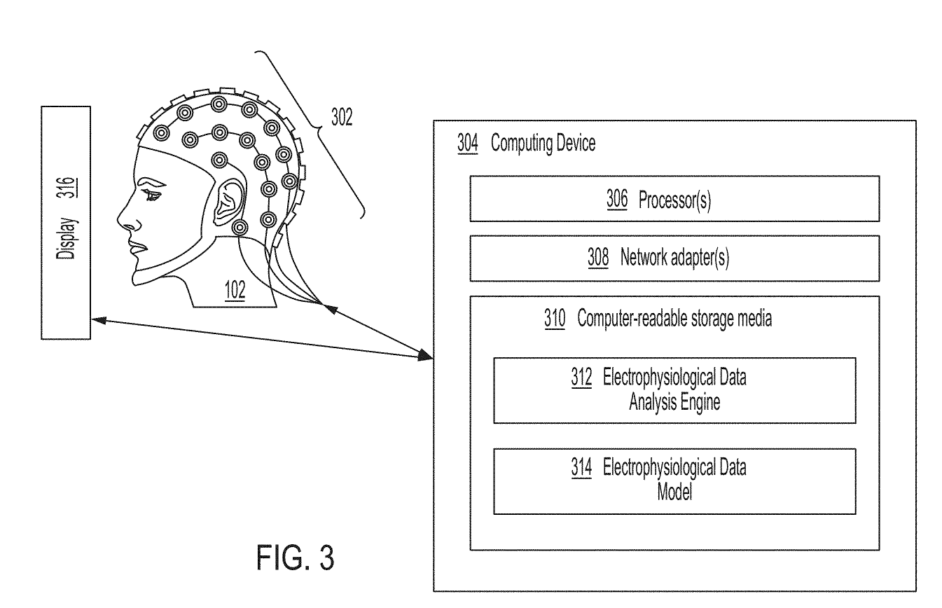

FIG. 3 is a diagram showing an exemplary system for monitoring and analyzing electrophysiological data of a subject 102, according to some embodiments. The system can be used to observe an analyze non-verbal markers of conditions associated with binocular rivalry, including to determine whether a subject exhibits signs associated with the condition, as discussed herein. The system includes an electrophysiological data capturing device 302 (also referred to herein as the capturing device 302), such as electrodes placed outside of the subject's scalp, implanted into the subject's brain, and/or the like. The electrophysiological data capturing device 302 is in communication with the computing device 304. The system also includes a display 316 in communication with the computing device 304, which can be configured to present the visual stimulus to the subject 102 for binocular rivalry testing. The computing device 304 includes one or more processors 306 configured to execute machine readable instructions configured to cause the one or more processors 306 to carry out the techniques described herein (e.g., which can be stored in the computer-readable storage media 310). The computing device 304 also includes one or more network adapters 308 configured to connect the computing device 304 with a network, such as the Internet (e.g., for Internet access, remote access, connection to remote electrophysiological data capturing devices, etc.). The computer-readable storage media 310 can include a number of different components, such as those shown in FIG. 3, including an electrophysiological data analysis engine 312 (also referred to herein as the analysis engine 312) and an electrophysiological data model 314 (also referred to herein as the data model 314).

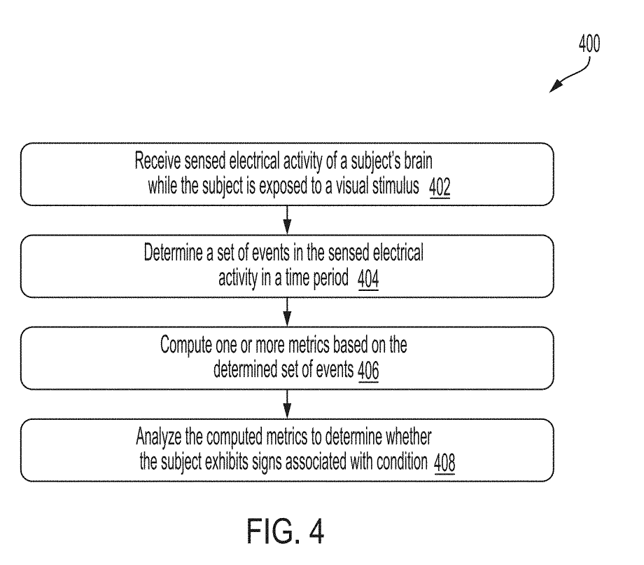

The analysis engine 312 comprises computer readable instructions that, when executed by the processor 306, analyze electrophysiological data received from the capturing device 302, e.g., as described further in conjunction with FIG. 4. As an illustrative example, the analysis engine 312 analyzes the received electrophysiological data to determine whether the data is indicative of the subject exhibiting signs that the subject likely has a condition, such as ASD and/or Fragile X syndrome.

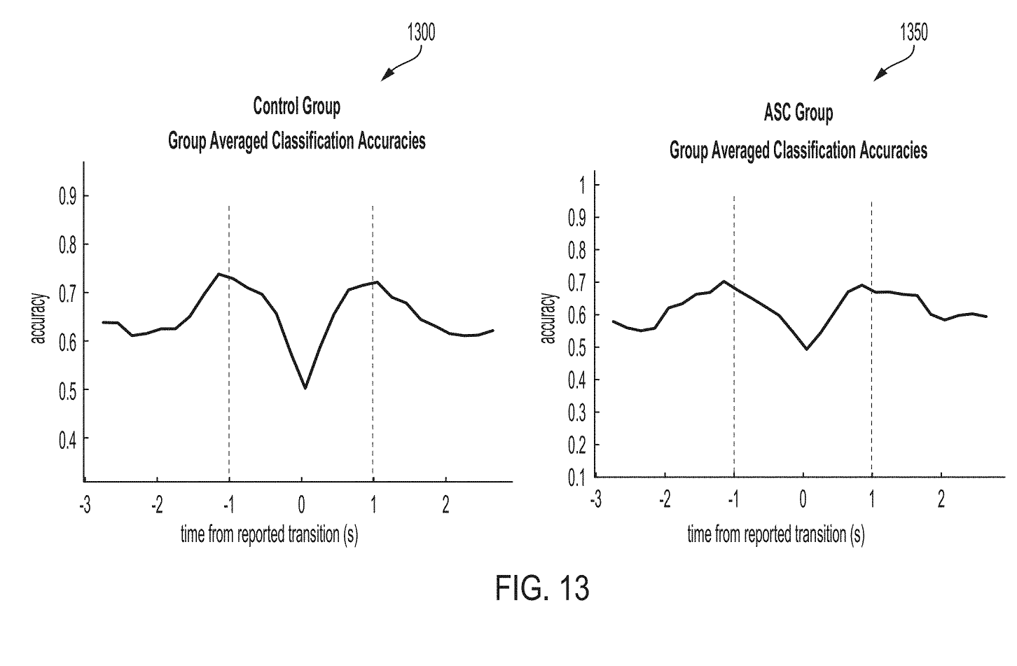

The data model 314 can be a machine learning data model, such as a support vector machine (SVM), that can be trained based on sample electrophysiological data for use by the electrophysiological data analysis engine 312. Once trained, the data model can be used to automatically analyze electrophysiological data to determine whether patients exhibit signs associated with a condition. The data model 314, including how it is trained and used, is discussed further below in conjunction with FIG. 13.

The display 316 can be, for example, a computer monitor (e.g., a CRT and/or LCD monitor) viewed through mirrors, a head-worn display (e.g., a VR headset), a smart phone, a tablet, a laptop screen, a television, and/or any other device sufficient to display the visual stimulus to the subject 102.

FIG. 4 is a flow diagram showing a computerized method 400 for analyzing electrophysiological data to determine whether a subject exhibits signs associated with a condition, according to some embodiments. The computerized method 400 can be executed by the processor 306 of the computing device 304 (e.g., and can be specified by machine readable instructions that are part of the electrophysiological data analysis engine 312 stored in the computer-readable storage media 310). At step 402, the computing device receives sensed electrical activity of a subject's brain while the subject is exposed to a visual stimulus (e.g., stimulus designed to trigger binocular rivalry, as discussed above). At step 404, the computing device determines a set of events in the sensed electrical activity in a time period. At step 406, the computing device computes one or more metrics based on the determined set of events. At step 408, the computing device analyzes the computed metrics to determine whether the subject exhibits signs associated with autism.

Referring to step 402, the sensed electrical activity can be received from the capturing device 302 discussed in conjunction with FIG. 3. The sensed electrical activity can comprise, for example, electroencephalogram (EEG) data, magnetoencephalography (ME) data, and/or the like. The sensed electrical activity can include sensed power in various frequency bands over time (e.g., in a sliding window of a certain timeframe, such as 100 milliseconds). The system can be configured to sense and/or analyze frequency bands associated with the frequency at which the images are presented to the subject. In some examples, the frequency bands include at least a first frequency band based on the frequency at which the first image is presented to the subject's left eye, and a second frequency band based on the frequency at which the second image is presented to the subject's left eye. FIG. 5 is a graph 500 showing an example of sensed frequency and power of EEG signals for a subject exposed to a visual stimulus. In the example shown in FIG. 5, the subject 102 is presented with the first image at 5.7 Hz and the second image at 8.5 Hz. The sensed electrical activity can be processed, e.g., using a Fourier transform, to determine the relation of frequency in Hz shown along the x-axis and power shown along the y-axis as graphed in graph 500. The graph 500 includes a spike at approximately 5.7 Hz, which corresponds to the neurons firing in response to the first image being presented at 5.7 Hz. The graph also includes a spike at approximately 8.5 Hz, which corresponds to the neurons firing in response to the second image being presented at 8.5 Hz. The data shown in FIG. 5 is the data from one subject of the trial (after Fourier transform); the trial is discussed further below in the Examples section.

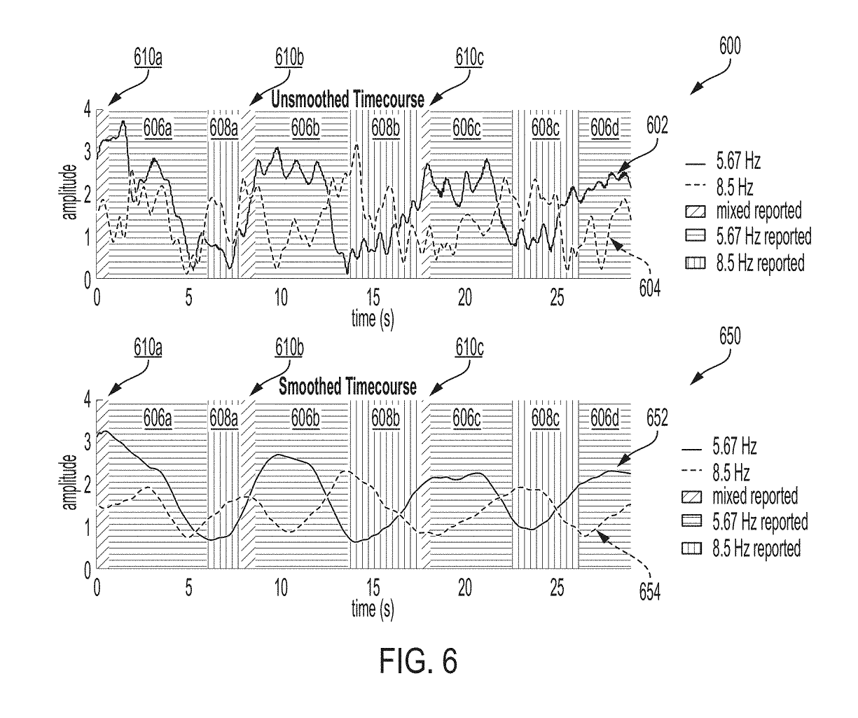

Referring further to steps 406 and 408 in FIG. 4, the electrophysiological data can be used to determine and analyze metrics associated with non-verbal markers (e.g., percept switch rates and/or depth of perception), in place of needing to obtain a verbal and/or physical response from the subject. FIG. 6 includes an unsmoothed graph 610 and a smoothed graph 650 of EEG signals overlaid on physical response information from a subject, according to some embodiments. Each graph includes a line (602, 652, respectively) showing the amplitude of the 5.67 Hz frequency (y-axis) over time (x-axis). Each graph also includes a line (604, 654, respectively) showing the amplitude of the 8.5 Hz frequency over time. Each graph further includes physical response information (e.g., a button presses) from the subject, including: (1) a physical response indicating perception of the first image being displayed at 5.67 Hz, shown by the shaded areas marked with 606a, 606b, 606c and 606d; (2) a physical response indicating perception of the second image being displayed at 8.5 Hz, shown by the shaded areas marked with 608a, 608b and 608c; and (3) a physical response indicating a mixed perception of both images, shown by the shaded areas marked with 610a, 610b and 610c. The data shown in FIG. 6 is the data from one subject of the trial (after Fourier transform and a time-frequency analysis); the trial is discussed further below in the Examples section.

These unsmoothed and smoothed graphs 610 and 650 in FIG. 6 show that when the subject is perceiving a particular image, the amplitude of the frequency associated with the currently-perceived image is higher than the amplitude of the frequency associated with the non-perceived image. Prior to the user's perception switching to the other image, the amplitude of the frequency associated with the currently-perceived image drops down, while the amplitude of the frequency associated with the other image increases.

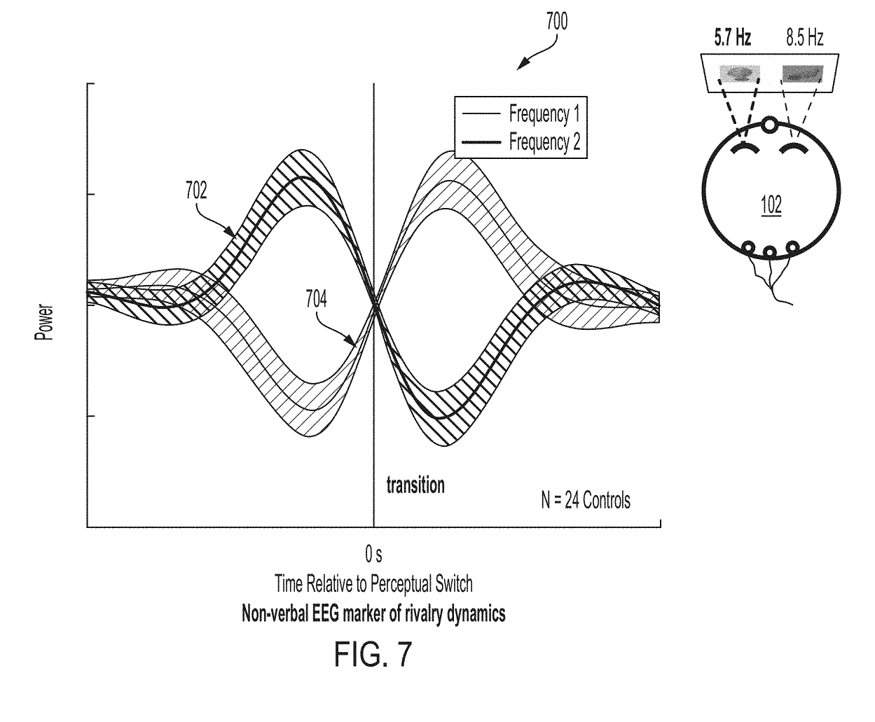

As can be seen in the graphs 610 and 650 in FIG. 6, repeating frequency patterns can be determined for perceptual switches. For example, FIG. 7 is a graph 700 showing averaged data for five seconds before, and five second after, a reported perceptual switch, according to some embodiments. The graph 700 includes a line 702 showing the power of the 5.7 Hz frequency (y-axis) from five seconds before the perceptual switch at time 0 seconds, and five seconds after the perceptual switch (x-axis). The graph 700 also includes a line 704 showing the power of the 8.5 Hz frequency over time from five seconds before the perceptual switch at time 0 seconds, and five seconds after the perceptual switch. For the perceptual switch associated with the data graphed in FIG. 7, the 5.7 Hz image is suppressed from perceptual awareness, and the 8.5 Hz image becomes dominant. This is consistent with the lines 702, 704 in graph 700, which show that the line 702 is suppressed down as the time crosses zero, while the line 704 becomes dominant as the time crosses zero. The data shown in FIG. 7 is the averaged data of twenty-four subjects of the control population of the trial; the trial is discussed further below in the Examples section.

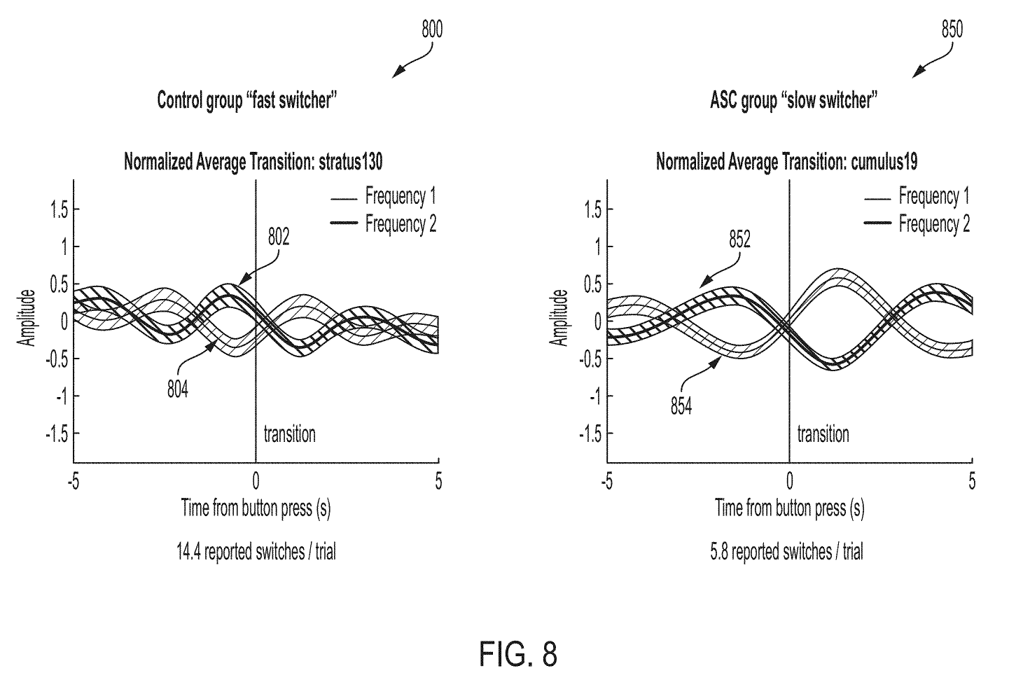

Referring further to step 406 in FIG. 4, one of the computed metrics can be a number of percept switches during a vision stimulus. FIG. 8 includes graphs 800, 850 of reported switches for a member of a control group and a member of an ASC group, according to some embodiments. Each graph includes a line (802, 852, respectively) showing the amplitude of the 5.7 Hz frequency (y-axis) over time (x-axis). Each graph also includes a line (804, 854, respectively) showing the amplitude of the 8.5 Hz frequency over time. The graph 800 includes data averaged across a series of binocular rivalry tests for a single individual from the control group of the trial discussed below, and the graph 850 includes data averaged across a series of binocular rivalry tests for a single individual in the autistic group of the trial. These graphs illustrate a portion of the number of switches for each subject: the subject in the control group reported an average of 14.4 perceptual switches per trial, whereas the subject in the autistic group reported an average of 5.8 switches per trial. Therefore, the switch period for the control data is shorter compared to the longer period for the ASC data.

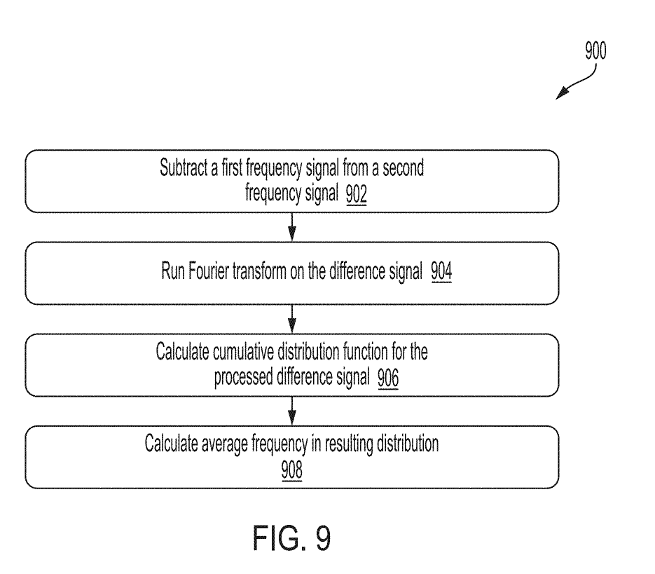

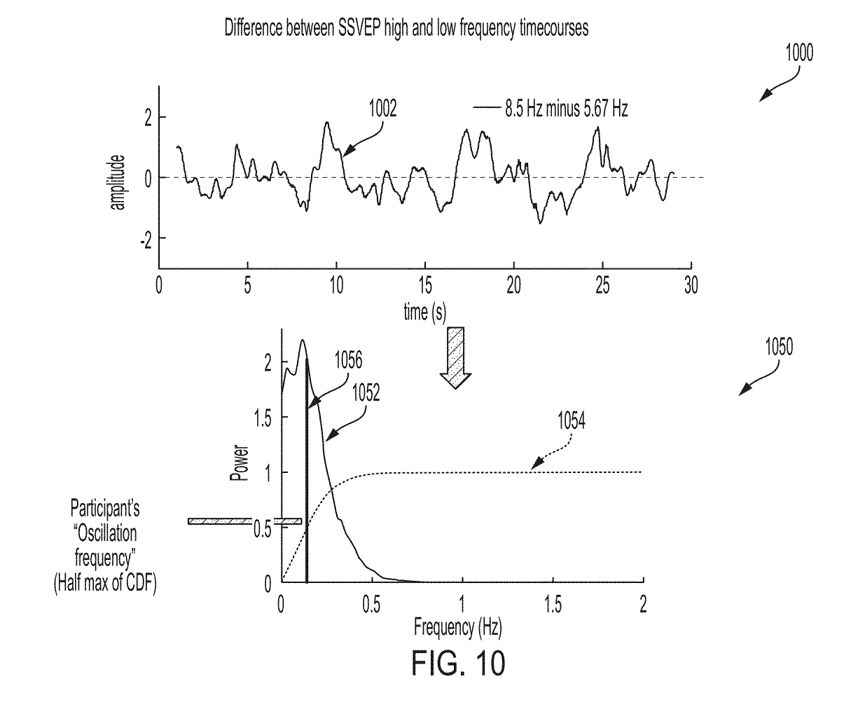

The electrophysiological data can be processed to determine other metrics associated with the subject. FIG. 9 is a flow diagram showing a computerized method 900 for calculating a subject's oscillation frequency, according to some embodiments. The method 900 can be used to quantify whether a subject is a “slow” or “fast” switcher in terms of binocular rivalry switches. FIG. 10 includes graphs 1000 and 1050 illustrating the calculation of a subject's oscillation frequency, according to some embodiments. Graph 1000 shows the amplitude (y-axis) of a difference signal 1002 over time (x-axis), calculated by subtracting the 5.67 Hz band signal from the 8.5 Hz band signal. Graph 1050 shows the Fourier transform of the difference signal 1002 (line 1052) to power (y-axis) over frequency in Hz (x-axis), the cumulative distribution 1054 of the Fourier transformed difference signal 1052, and the half maximum of the cumulative distribution 1056.

Referring to both FIGS. 9 and 10, at step 902, the computing device subtracts a first frequency signal (e.g., the 5.67 Hz band) from the second frequency signal (the 8.5 Hz band) to generate the difference signal 1002. As a result of the difference calculation, the perception switching is now represented when the signal crosses zero. In this example, 0 represents a switch, a negative amplitude is associated with brain activity caused by the 8.5 Hz image, and a positive amplitude is associated with brain activity caused by the 5.7 Hz image. At step 904, the computing device runs a Fourier transform on the difference band 1002 to generate the Fourier transformed difference signal 1052. At step 906, the computing device runs a cumulative distribution function on the Fourier transformed difference signal 1052 to generate the cumulative distribution 1056. At step 908, the computing device calculates the “average” frequency in the resulting distribution (e.g., the half maximum of Cumulative Distribution Function). In the example shown in FIG. 10, the subject's calculated average frequency is approximately 0.20 Hz.

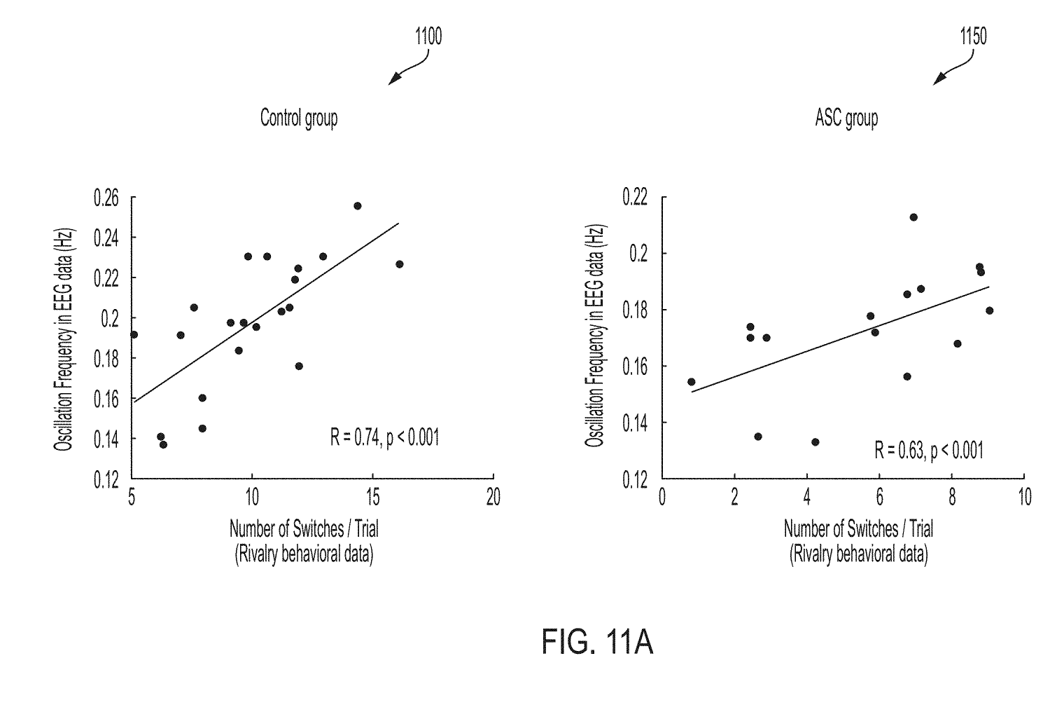

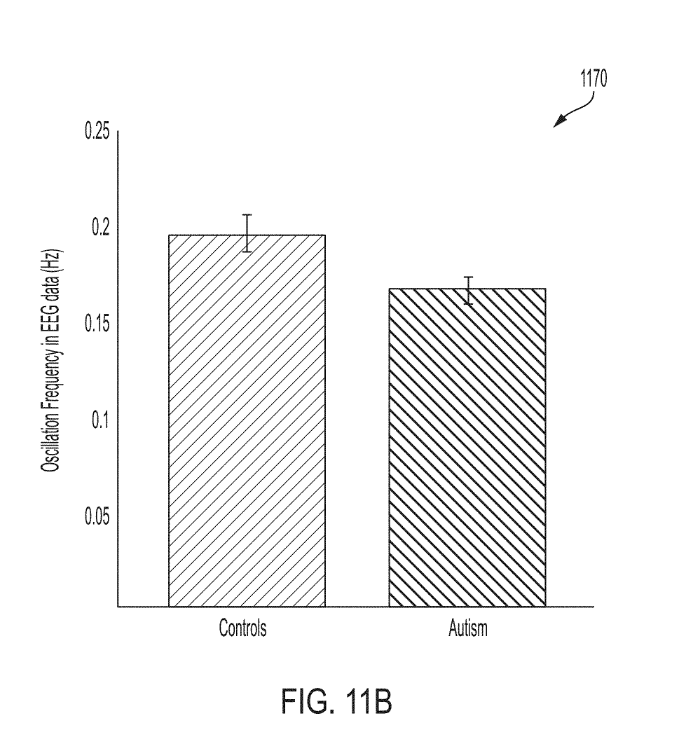

The calculated average frequency can represent a subject's neurally derived switch rate, or speed of switching. FIG. 11A includes graphs comparing the number of switches per trial (x-axis) with the oscillation frequency (y-axis) for the control group (graph 1100) and the autistic group (graph 1150), according to some embodiments. At a high level, these graphs 1100 and 1150 show how fast the group subjects are oscillating, on average. FIG. 11B includes a graph 1170 that shows the mean of the oscillation frequency seen in the electrophysiological data plotted in graphs 1100, 1150, for the control group and the autistic group. Graph 1170 shows that the mean oscillation frequency is approximately 0.20 Hz for the control group and approximately 0.16 Hz for the ASC group. Therefore, the exemplary calculated frequency of approximately 0.20 Hz indicates that the subject is likely not a member of the ASC group (and therefore likely does not exhibit signs of autism). “R” in these graphs refers to a subject's correlation coefficient, e.g., how correlated on a scale from 0 to 1 the EEG-determined switch rates are with the behaviorally-reported switch rates.

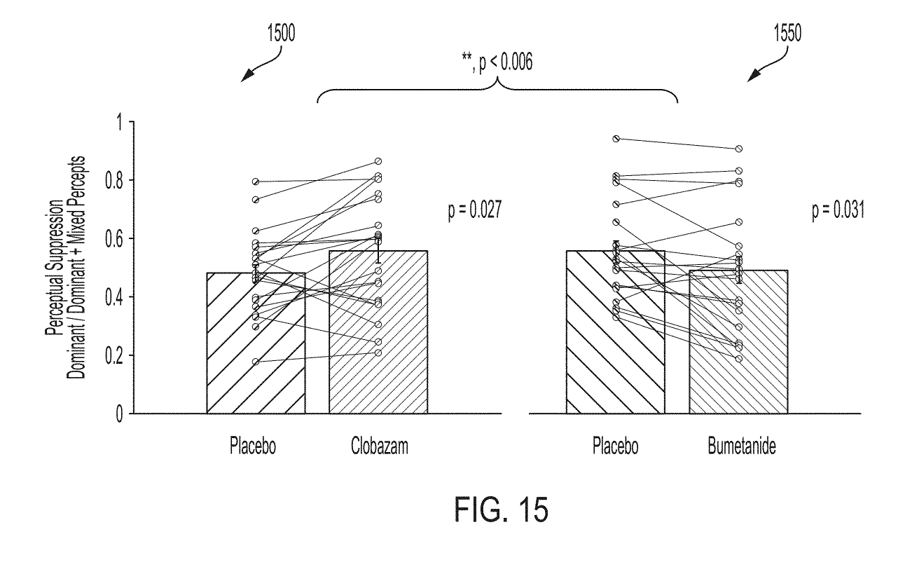

As discussed further in conjunction with FIG. 15, another non-verbal marker that can be measured is the depth of perceptual suppression, which represents how long the subject perceives one of two images compared to perceiving a mix of the two images. Therefore, the techniques discussed herein can be used to determine a subject's switch rate, depth of perceptual suppression, and/or other metrics that analyze the subject's brain activity while being exposed to a binocular rivalry visual stimulus. For example, a weaker depth of perception can be indicative of autism.

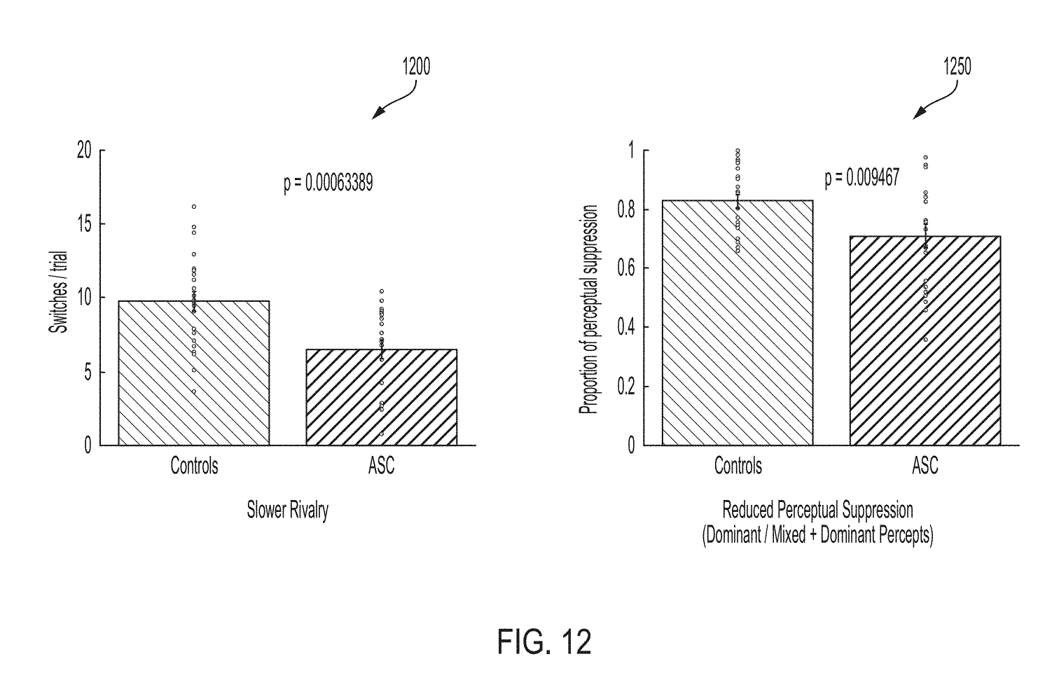

FIG. 12 shows bar graphs 1200, 1250 comparing the number of switches per trial in the first graph with the proportion of perceptual suppression (e.g., dominant/dominant+mixed percepts) in the second graph, according to some embodiments. Graph 1200 compares the number of switches per trial for the control and ASC groups, showing that the median for the control group is approximately 10 switches per trial, while the median for the ASC group is approximately 7 switches per trial. Graph 1250 compares the proportion of perceptual suppression for the control and ASC groups, showing that the median for the control group is a proportion of perceptual suppression of approximately 0.8, while the median for the ASC group is a proportion of perceptual suppression of approximately 0.7. In this example, the values in these graphs are derived from the behaviorally-reported data (e.g., the x-axis of FIG. 11A). These graphs show the subject's neural behavioral data, including the subject's switch rate and proportion of perceptual suppression, which is similar to physical response data.

As demonstrated above and further below, the techniques can be used to determine a behavioral marker of autism using a subject's neural signature (e.g., slower rivalry dynamics are indicative of the subject exhibiting signs associated with autism). The techniques can be used to classify the subject's perceptual state from the subject's neural signature.

In some embodiments, machine learning techniques are used to build a classification model (e.g., a support vector machine classifier) used to classify a subject's perceptual state. The model can be built based on certain non-verbal markers associated with the condition being tested. For example, for autism, the model can be built based on non-verbal markers such as the speed of rivalry and/or changes in the amplitude of physiological data associated with perceptual events, as discussed above.

In some embodiments, the model can be trained on training data in which the known flicker pattern is recorded and used to build the model. Therefore, the model can be built using data associated with known testing patterns to train the model to recognize the switch from images (e.g., from 5.7 Hz to 8.5 Hz, and/or vice versus). The model can then be used to analyze a subject's actual electrophysiological data.

For example, recursive least squared segments can be used to train a classifier, such as by using a time-frequency analysis of training or simulation data. A combination of various features can be used when training the model, such as various metrics (e.g., the amplitude of the frequency over time, the 1^{st} derivative of the data, and/or the 2^{nd} derivative of the data), various frequencies (e.g., the principle frequencies used to tag the images, harmonics of those frequencies, and/or intermodulation data), and/or various numbers of occipital channels. For example, seven occipital channels can be used, such as different plugs as illustrated in FIG. 3, some being on the right hemisphere and some being on the left hemisphere.

In some embodiments, the classifier can be configured to classify data at certain times, e.g., a certain time period before and/or after a potential measured (or reported) perceptual transition. FIG. 13 includes graphs 1300, 1350 showing the accuracy of the analysis techniques compared to the time of a subject's reported transition, according to some embodiments. Both graphs 1300 (for the control group) and 1350 (for the ASC group) show that the average classification accuracy is the poorest at around 0 seconds, which is the time of the perceptual transition. Both graphs 1300, 1350 similarly illustrate that a strong classification accuracy occurs around one second before and after the perceptual transition. Therefore, as shown in this example, the classifier can be configured to classify data one second before and/or one second after the transition. In some embodiments, cross-validation can be used to train the model, such as using a leave-one-out cross-validation to train the model.

In some aspects, provided herein is a method of evaluating a subject. A method of evaluating a subject comprises providing a visual stimulus to the subject and detecting brain activity of the subject according to any one of the methods disclosed herein to determine a metric. A subject is likely to have a condition that is associated with binocular rivalry if the metric is less than a reference metric. In some embodiments, a metric for a subject is less than a reference metric by at least 1% of the reference metric (e.g., at least 1%, 2%, 5%, 10%, 20%, 50%, 80%, 90%, 95%, 99% or 99.9%).

In some embodiments, a method of evaluating a subject further comprises clinically evaluating the subject to determine if the subject has an ASD. A subject may be clinically evaluated for symptoms and criteria set forth in the Diagnostic and Statistical Manual of Mental Disorders (DSM).

In some embodiments, a subject is evaluated by any one of the methods disclosed herein at more than one points in time to determine the prognosis or development of the condition or the subject, if the subject is a child.

In some aspects, provided herein is a method of determining the efficacy of a treatment in a subject suffering from a condition that is associated with binocular rivalry. In some embodiments, a method to determine the efficacy of a treatment comprises providing a visual stimulus to the subject, and detecting brain activity of the subject according to any one of the methods disclosed herein to determine a metric, wherein the treatment is said to be efficacious if the metric is increased after the administration of the treatment. In some embodiments, a metric is increased in a subject after the administration of treatment by at least 1.1 times (e.g., at least 1.1, 1.2, 2, 5, 10, 20, 30, 40, 50, 60, 80, 90, 95, 100, 150, 200, 500 or 1000 times). In some embodiments, a metric is increased in a subject after the administration of treatment by at least 1.1 times (e.g., 1.1-1000, 1.1-2, 2-5, 2-10, 5-20, 10-50, 20-100, 100-1000 times). In some embodiments, a metric is increased in a subject after the administration of treatment by at least 1% (e.g., by at least 1-10, 10-20, 10-50, 20-60, 30-70, 50-80, 50-90, 60-100, 100-200, 100-1000%).

Provided herein are also methods used to assay compounds to identify efficacious drugs for conditions associated with binocular rivalry.

Provided herein is also a method of treating a subject diagnosed with a condition that is associated with binocular rivalry. A method of treatment as provided herein may comprise providing a visual stimulus, or directing or ordering a test to provide a visual stimulus to a subject to determine a metric according to any one of the methods disclosed herein, and administering a treatment to a subject if the subject's metric is less than a reference metric. In some embodiments, a method of treating a subject diagnosed with a condition that is associated with binocular rivalry comprises selecting the subject on the basis that the subject has a metric that is less than a reference metric, and administering a therapeutic treatment to the subject.

In some embodiments of any one of the methods disclosed herein, a metric is an average perceptual event rate. The average perceptual event rate can be, for example, the average number of transitions from perceiving one image to another image, and/or perceiving a mix of the two images. In some embodiments, a metric is perceptual suppression based on a ratio of dominant to mixed percepts; or both.

As used herein, a “reference metric” is a value of a metric that is used to set a threshold to decide whether a subject being evaluated is likely to have a condition that is associated with binocular rivalry. In some embodiments, a reference metric is obtained using metric measurements on a reference population. In some embodiments, a reference metric is the average of the measurements of the metrics of all the individuals in a reference population. In some embodiments, a reference metric is a certain percentile (e.g., 50^{th} percentile, 80^{th} percentile or 90^{th} percentile) of the measurements of the metrics of all the individuals in a reference population. In some embodiments, a reference population comprises individuals of the same species as a subject being evaluated. In some embodiments, a reference population comprises a test species. For example, a reference population of lab mice may be used to evaluate a human subject. In some embodiments, a cross-species comparison involves subject of the different species having similar genetic mutations, or mutations in the same gene or gene locus. In some embodiments, reference population consists of individuals that do not suffer from a condition that is associated with binocular rivalry. In some embodiments, reference population consists of individuals that do not suffer from an ASD. In some embodiments, reference population consists of individuals that do not suffer from any mental disorder.

In some embodiments of any of the methods disclosed herein, a visual stimulus comprises presenting to the subject a first frequency-tagged image to the left eye and a second frequency-tagged image to the right eye, wherein the frequency of the first frequency-tagged image is different from the frequency of the second frequency-tagged image. In some embodiments, the first image is presented exclusively to the left eye and second image is presented exclusively to the right eye. In some embodiments, this is achieved using a stereoscope or mirror, or a lens. In some embodiments, the images are displayed on a monitor (e.g., a digital monitor) or a headset. In some embodiments, the images are presented to a subject so that the subject can see the images as they look straight ahead.

As disclosed herein, a condition that is associated with binocular rivalry is any condition or disease in which a subject manifests or is suspected to manifest symptoms of binocular rivalry (e.g., experiencing a reduction of superimposition of two different images presented to two eyes of the subject). In some embodiments, a condition that is associated with binocular rivalry is an Autism Spectrum Disorder (ASD). In some embodiments, an ASD is one or more condition in the following group: Autistic Disorder, Asperger's Disorder, Rett's Disorder, Childhood Disintegrative Disorder, and Pervasive Developmental Disorder Not Otherwise Specified (PDD-NOS). In some embodiments, an ASD is a condition that is classified as such in the Diagnostic and Statistical Manual of Mental Disorders (DSM). In some embodiments, the DSM is DSM-IV. In some embodiments, the DSM is DSM-V. In some embodiments, a condition that is associated with binocular rivalry is Fragile X syndrome.

Aspects of the disclosure relate to methods for use with a subject, such as human or non-human primate subjects. In some embodiments, the subject is a human subject. In some embodiments, a human subject is a child (e.g., an infant). In some embodiments, a subject is a child who is still developing certain skills (e.g., language, or other social skills). In some embodiments, a subject is a child who is still developing certain skills (e.g., language, or other social skills) and is clinically showing deficits in those skills. In some embodiments, a subject showing deficits in certain skills (e.g., language, or social skills) is an adult. In some embodiments, a subject does not manifest any developmental deficits but is part of a study to evaluate a diagnostic method or the safety and/or efficacy of a treatment. In some embodiments, a subject is non-verbal. In some embodiments, a non-verbal subject is someone who does not speak at all. In some embodiments, a non-verbal subject is someone who uses a few words in a meaningful manner but are unable to carry one any kind of significant conversation. For example, a non-verbal subject may say “car” to mean “let's go somewhere.” In some embodiments, a non-verbal subject does not use spoken language effectively but is able to communicate with written or typed language, picture cards, or sign language. In some embodiments, a subject is high-functioning. In some embodiments, a high-functioning subject is someone who is deemed to be cognitively higher functioning compared to other people with ASD. In some embodiments, high-functioning subjects have an intelligence quotient of 70 or more.

In some embodiments, a subject as described herein has been previously diagnosed to have a mental disorder. Examples of a mental disorder include but are not limited to an ASD (as described above), Fragile X syndrome, an anxiety disorder, attention deficit hyperactivity disorder (ADHD), a sensory processing disorder, or Social (Pragmatic) Communication Disorder. In some embodiments, the mental disorder with which a subject has been previously diagnosed is associated with symptoms that overlap with an ASD or Fragile X syndrome.

Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, a subject is a lab animal (e.g., a rodent or a non-human primate, or a bird). A lab animal may be a mouse, a rat, or a ferret. Other exemplary subjects include domesticated animals such as dogs and cats; livestock such as horses, cattle, pigs, sheep, goats, and chickens; and other animals such as mice, rats, guinea pigs, and hamsters.

Aspects of this disclosure relate to a treatment for a condition that is associated with binocular rivalry. In some embodiments, a treatment is experimental. A treatment can comprise a behavioral therapy or administration of a pharmaceutical agent. A behavioral therapy may be teaching of a developmental skill. In some embodiments, a treatment is a standard of care for the particular condition that is associated with binocular rivalry. For example, for a subject with an ASD, a treatment may include administration of a pharmaceutical agent that is a selective serotonic reuptake inhibitor or a gamma-Aminobutyric acid (GAB A) receptor modulator. Non-limiting examples of serotonin reuptake inhibitors are citalopram, fluoxetine or sertraline. A GABA receptor modulator may be a GABA receptor agonist or a GABA receptor positive allosteric modulator. A GABA receptor modulator may act on GABA_{A}, GABA_{B}, GABA_{A-ρ} or GABA_{C}. In some embodiments, a GABA receptor agonist is clobazam or arbaclofen. Other non-limiting GABA modulators are Barbiturates (e.g., phenobarbital), Bamaluzole, GABA, Gabamide gamma-Amino-beta-hydroxybutyric acid, Gaboxadol Ibotenic acid, Isoguvacine Isonipecotic acid, Muscimol, Phenibut, Picamilon, Progabide, Propofol, Quisqualamine, SL 75102 Thiomuscimol, 1,4-Butanediol, Baclofen, R-baclofen, gamma-Butyrolactone (GBL), gamma-Hydroxybutyric acid (GHB), gamma-Hydroxyvaleric acid (GHV), gamma-Valerolactone (GVL), Lesogaberan, Tolgabide, (Z)-4-Amino-2-butenoic acid, N⁴-Chloroacetylcytosine arabinoside, ivermectin, Benzodiazepines (e.g., diazepam, alprazolam), Carbamates (e.g., meprobamate, carisoprodol), Chloralose, Chlormezanone Clomethiazole, Dihydroergolines (e.g., ergoloid (dihydroergotoxine)), Etazepine Etifoxine, Imidazoles (e.g., etomidate), Kavalactones, Loreclezole, Neuroactive steroids (e.g., allopregnanolone, ganaxolone), Nonbenzodiazepines (e.g., zaleplon, zolpidem, zopiclone, eszopiclone), Petrichloral, Phenols (e.g., propofol), Piperidinediones (e.g., glutethimide, methyprylon), Propanidid, Pyrazolopyridines (e.g., etazolate), Quinazolinones (e.g., methaqualone), Sulfonylalkanes (e.g., sulfonmethane, tetronal, trional), and Valerian constituents (e.g., valeric acid, valerenic acid). It is to be understood that a GABA modulator may be various enantiomeric forms of compounds listed herein.

In some embodiments, a the pharmaceutical agent affects irritability and aggression, aberrant social behavior, hyperactivity and inattention, repetitive behaviors, cognitive disorders, or insomnia. Non-limiting examples of pharmaceutical agents affecting irritability and aggression are Risperidone, Aripiprazole, Clozapine, Haloperidol, or Sertraline. Non-limiting examples of pharmaceutical agents affecting aberrant social behavior are Risperidone, Haloperidol, Oxytocin, or Secretin. Non-limiting examples of pharmaceutical agents affecting hyperactivity and inattention are Methylphenidate or Venlafaxine. Non-limiting examples of pharmaceutical agents affecting repetitive behaviors are Fluoxetine, Citalopram, or Bumetanide. Non-limiting examples of pharmaceutical agents affecting cognitive disorders are Memantine or Rivastigmine. Non-limiting examples of pharmaceutical agents affecting insomnia are Mirtazapine or Melatonin.

In some embodiments, “administering” or “administration” means providing a material to a subject in a manner that is pharmacologically useful.

To “treat” a disease as the term is used herein, means to reduce the frequency or severity of at least one sign or symptom of a condition experienced by a subject. The pharmaceutical agents described above or elsewhere herein are typically administered to a subject in an effective amount, that is, an amount capable of producing a desirable result. The desirable result will depend upon the active agent being administered. A therapeutically acceptable amount may be an amount that is capable of treating a condition, e.g., an ASD. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

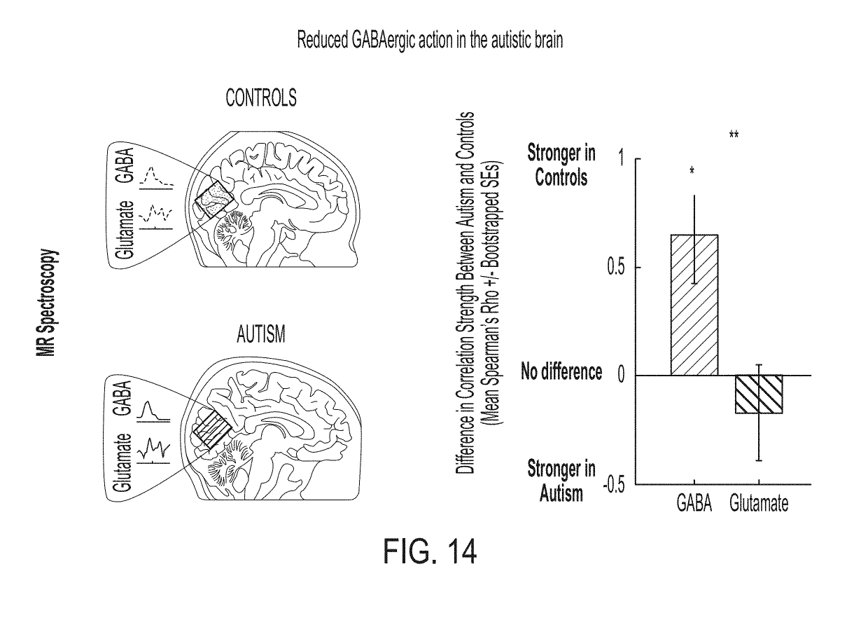

FIG. 14 is a diagram illustrating the reduced GABAergic action in the autistic brain, according to some examples. The panel on the left depicts magnetic resonance spectra acquired from individuals with and without ASD using the visual cortex region as the acquisition region, which include neurotransmitters that are predicted to govern binocular rivalry dynamics from computational models (e.g., GABA and glutamate). The right panel shows that GABA shows a significantly stronger effect on rivalry dynamics in controls, as compared to those with ASD, demonstrating a selective disruption in GABAergic action in the autistic brain. Error bars represent 1 SEM.

FIG. 15 demonstrates that the methods to measure the depth of perceptual suppression as disclosed herein can be used to measure response of the GABAergic pathway to pharmaceutical agents. FIG. 15 shows that the depth of perceptual suppression is greater in subjects treated with GABA agonist clobazam compared to those that were treated with a placebo agent, whereas the depth of perceptual suppression is greater in subjects treated with bumetanide compared to those that were treated with a placebo agent.

The techniques discussed herein can be used to determine the depth of perceptual suppression for subjects, measured as the ratio of dominant percepts (e.g., when an image is dominant in perception over the other image) over the sum of dominant and mixed percepts (e.g., when the subject perceives a mix of both images).

### EXAMPLES

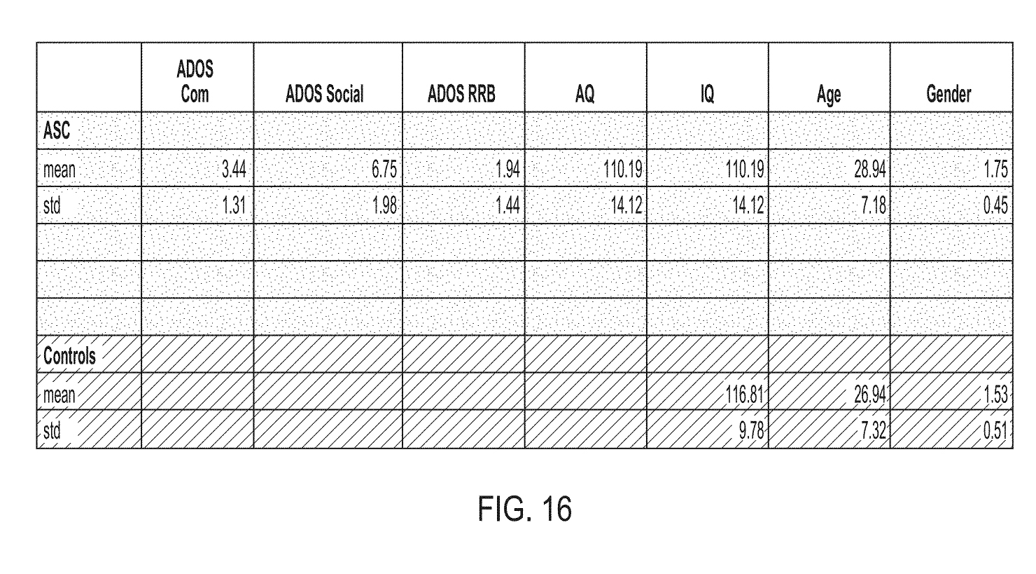

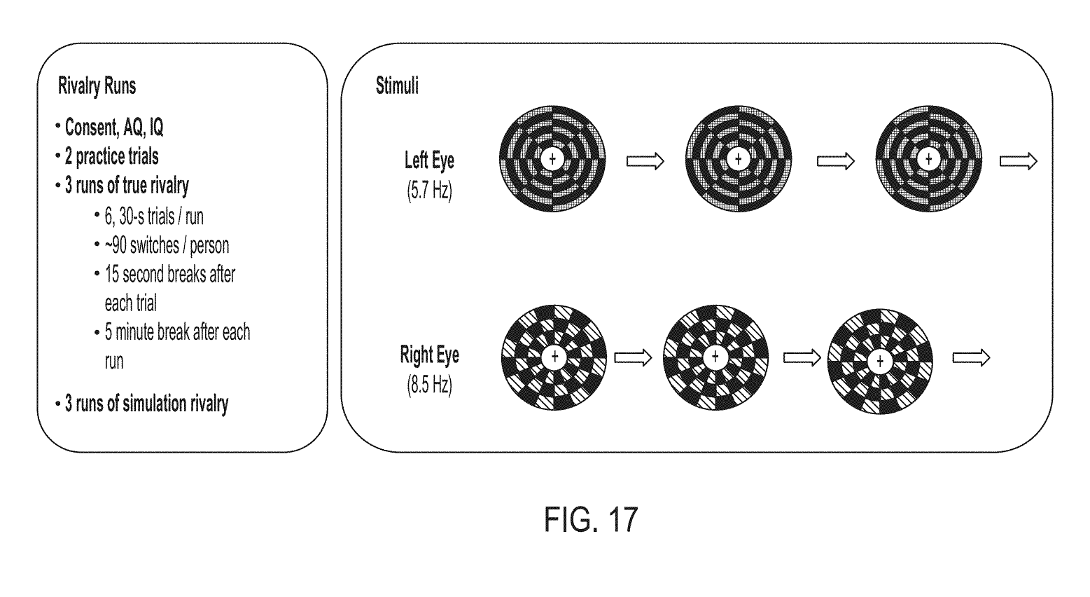

As noted above, FIGS. 2, 10-13 and 15 graphically depict exemplary data obtained during the first exemplary study discussed in this Examples section. FIGS. 16-17 illustrate aspects of the first exemplary study used to analyze subjects using the techniques discussed above, according to some embodiments. As discussed further below, the first exemplary study confirms that markers of autism can be obtained from a subject's neural responses in a controlled and predictable manner, with high accuracy.

FIG. 16 is a table that illustrates the subjects used in the study, which started with 23 control subjects and 23 autistic subjects, ranging in age from 16-40 years of age, matched for age and IQ. A specialized clinician judged that the patients met international diagnostic criteria for autism according to the Diagnostic and Statistical Manual of Mental Disorders, 4th edition (DSM-IV), which is hereby incorporated by reference herein in its entirety. The patients had normal or corrected-to-normal vision and individuals with other psychiatric conditions, such as attention deficit-hyperactivity disorder, were not recruited. Written consent was obtained from all participants in accordance with a protocol approved by the Massachusetts Institute of Technology Committee on the Use of Humans as Experimental Subjects.

Subjects with and without autism were matched for age, gender non-verbal IQ, as evaluated using the Wechsler Abbreviated Scale of Intelligence. Subjects also completed the Autism Spectrum Quotient (AQ), a self-report questionnaire that quantifies autistic traits across both autistic and control populations (Baron-Cohen et al., “The autism-spectrum quotient (AQ): evidence from Asperger syndrome/high-functioning autism, males and females, scientists and mathematicians,” 2001, which is hereby incorporated by reference herein in its entirety). Additionally, an hour-long diagnostic protocol was administered to all autistic subjects (the Autism Diagnostic Observation Schedule (ADOS); Lord et al., 2000, which is hereby incorporated by reference herein in its entirety).

The initial population was refined, e.g., to remove candidates with low SNRs, to account for age and IQ matching. The two groups used for the study were as follows: 17 control subjects in the control group, and 16 ASCs in the ASC group. The table shown in FIG. 16 shows the mean and standard deviation of various parameters, including the Autism Diagnostic Observation Schedule (ADOS) communication assessment (ADOS Com), the ADOS social interaction assessment (ADOS Social), the ADOS Restricted Repetitive Behaviors (ADOS RRB), Autism Spetrcum Quotient (AQ) (e.g., the ability to respond to change and challenges), Intelligences Quotient (IQ), Age and Gender.

FIG. 17 illustrates the study protocol and associated visual stimulus. The study protocol which included 2 practice trials, 3 runs of true rivalry, and 3 runs of simulation rivalry, which are described further below. Participants viewed a computer monitor (a Dell CRT) from a distance of 50 cm (fixed using a chin rest) through a mirror stereoscope. The stereoscope reflected the left and right sides of the monitor into the participants' left and right eyes, so that each eye was presented with only one of the two images (red or green, as shown in FIG. 17). Fusion was achieved for each participant individually before the experiment began by slowly moving two black circles from the edge toward the center of the screen until the point at which participants reported seeing one circle. All testing took place in a darkened, shielded room.

For the true runs of rivalry, for each trial, participants viewed the two high-contrast “dartboard” stimuli, one red and one green as shown in FIG. 17, one on the left and one on the right side of the screen. Each dartboard (average width and average height: 2 degrees visual angle) appeared on the horizontal meridian of a yellow screen, surrounded by a black circle to support binocular fusion (radius: square root of two), and centered on a black fixation cross. The side of the screen on which a red or green dartboard was presented was counterbalanced across trials. The green dartboard was frequency tagged at 5.7 Hz and the red dartboard was frequency tagged at 8.5 Hz.

Before the experiment, participants were given instruction with the task and practiced two experimental trials. Specifically, participants were instructed to continuously indicate whether they perceived the red image, the green image, or a mixture of the two images.

Testing sessions were composed of two 15 second practice trials, followed by three blocks of six 30 second experimental trials. Each run was separated by a 15 second break, which was indicated by a dimming of the screen background. At the beginning of each trial, the black circles appeared and participants controlled dartboard onset by pressing the “Up” key to start the trial.

Participants were asked to continuously report whether they perceived a fully dominant percept—the red dartboard (right key) or the green dartboard (left key)—or a mixture of the two (up key).

For the simulation rivalry, following the experimental runs, participants performed three blocks of six 30 seconds simulation rivalry trials. Simulation trials were identical to the main experiment, except that, in the simulation condition, two identical frequency-tagged dartboards were presented to the two eyes at all times (either red or green). Participants viewed stepwise, sudden transitions between these two single-images, which alternated on a randomized time schedule. The average image duration was 1.9 seconds, based on the average percept duration during a previous rivalry experiment from our lab (Robertson et al., Slower Rate of Binocular Rivalry in Autism, 2013, which is hereby incorporated by reference herein in its entirety). This stepwise, sudden transition allowed us to: 1) determine whether there were any differences in participants' response latencies to clear, obvious transitions, and 2) measure average EEG response to fully-on and full-off stimuli in each frequency band. Participants were not informed that they were viewing an alternating stimulus rather than a rivalry stimulus, and completed the same task that they performed for the experimental trials.

Regarding the EEG setup, EEG data were recorded on a Biosemi Active Two System with a lowpass filter at 0.16 Hz, a highpass filter at 100 Hz, and a sampling frequency of 512 Hz. For each participant, data was collected from 32 posterior channels using a 64-channel Ag—AgCl electrode cap. While 1 channels (Oz) were used for the naïve Fourier analysis and time frequency analysis, 7 channels were used for the machine learning analysis, including Oz. The electrodes were soaked in a mild saline solution for ten minutes prior to each use, allowing electrode impedances to settle more quickly before measurement began.

EEG data was pre-processed using the FieldTrip MATLAB toolbox (REF) and custom MATLAB code. Raw data for each trial was first linearly detrended, high-pass filtered at 2 Hz, and bandstop filtered at 59-61 Hz. Electrodes were then re-referenced using the common average. Noisy electrodes were identified visually using histograms of kurtosis, mean, and variance for each trial, and outliers were discarded on a trial-by-trial basis. If an electrode was determined to be an outlier in more than half of the trials in a run, that run was eliminated altogether.

To track the amplitudes of frequencies of interest over time, whether it be the tagging frequencies, their harmonics, or their intermodulation frequencies, pre-processed EEG data was first passed through a bandpass filter+/−0.5 Hz around the desired frequency. An adaptive recursive least square (RLS) filter (e.g. Tang and Norcia, “An adaptive filter for steady-state evoked responses,” 1995, which is hereby incorporated by reference herein in its entirety) was then used to extract amplitude timecourses for that frequency. The first second of each timecourse was discarded to eliminate the transient spike in amplitude corresponding to stimulus onset.

The SNR was calculated for each 30 second trial using the fast Fourier transform (FFT) for Oz (e.g., the electrode which falls on the midline (between hemispheres) over the posterior pole of occipital cortex, such that it hovers above the primary visual region of the brain, V1). SNR was then computed separately for each tagging frequency as the power at that frequency divided by the mean power in a surrounding band of noise frequencies (+/−0.5 Hz). EEG data for a given trial was only used in further analysis if the SNR of both tagging frequencies exceeded a threshold of 2.

To assess the frequency at which a participant oscillates between percepts during rivalry, the amplitude time courses were analyzed using the fast Fourier transform (FFT). This method provided a measure of switch rate completely naïve to the participant's behavioral report, as discussed above.

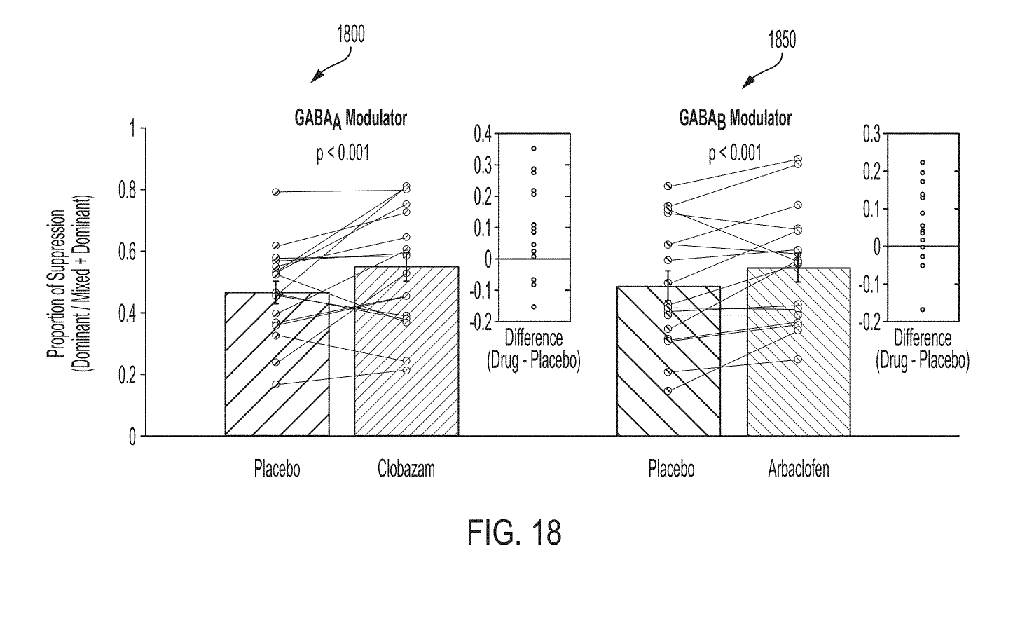

FIGS. 18-20 graphically depict exemplary data obtained during a second exemplary study. Consistent with the first study, and as discussed further below, the second exemplary study further confirms that markers of a mental disorder (e.g., ASC) can be obtained from a subject's neural responses in a controlled and predictable manner, with high accuracy. In particular, the second exemplary study further confirms the causal, mechanistic link between the GABA pathway and perceptual suppression during rivalry.

In the second exemplary study, thirty-four adult participants, ranging in age from 16-40 years of age, participated in two separate studies. The first study investigated the effects of the GABA_{A} modulator clobazam on binocular rivalry dynamics, and the second study investigated the effect of the GABA_{B} modulator arbaclofen on binocular rivalry dynamics. Each study took place over three days and was conducted in a counter-balanced (double)-blind design. The first day involved a health screening and practicing rivalry through seven 45-second trials. The second day involved administering the drug and placebo, performing a drowsiness survey after fifty-five minutes from administration, and performing the rivalry experiment sixty minutes from administration. The third day similarly involved administering the drug and placebo, performing a drowsiness survey after fifty-five minutes from administration, and performing the rivalry experiment sixty minutes from administration. The third day concluded with a post-study survey. The rivalry experiment for each of the second and third days included two practice trials, two blocks of experimental trials (each block consisting of five forty-five second trials), and two blocks of control trials (each block consisting of three forty-five second trials). The drowsiness survey showed that clobazam increased drowsiness in participants, while arbaclofen did not.

FIG. 18 further demonstrates that methods to measure the depth of perceptual suppression as disclosed herein can be used to measure the response of the GABAergic pathway to pharmaceutical agents. FIG. 18 includes two graphs 1800 and 1850 comparing the depth of perceptual suppression (dominant/dominant+mixed percepts) for a placebo and clobazam and arbaclofen, respectively. Graph 1800 shows that the depth of perceptual suppression is greater in subjects treated with GABA_{A} agonist clobazam compared to those that were treated with a placebo agent, and graph 1850 shows that the depth of perceptual suppression is greater in subjects treated with GABA_{B} agonist arbaclofen compared to those that were treated with a placebo agent. Thus, as shown in FIG. 18, both GABAergic drugs exerted a strong and specific effect on perceptual suppression during rivalry.

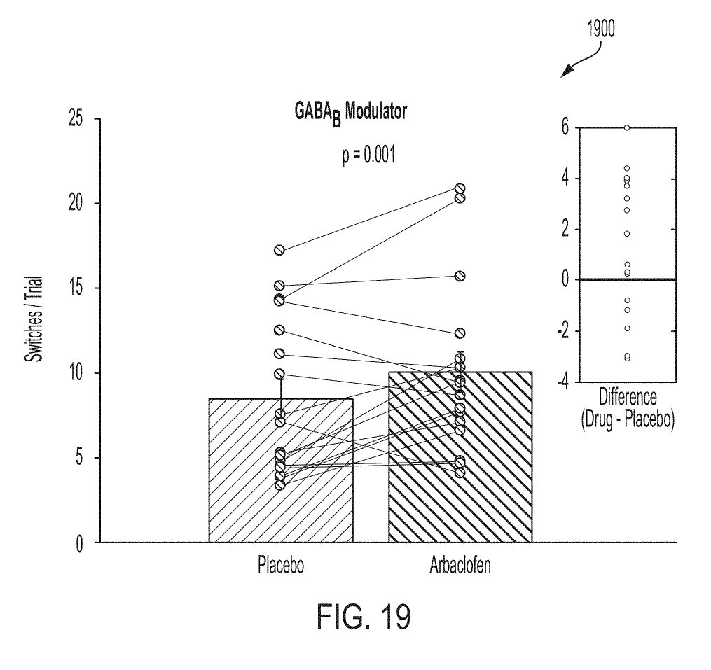

FIG. 19 shows a bar graph 1900 comparing the number of switches per trial for subjects treated with a placebo agent and arbaclofen, according to some embodiments. As shown in the bar graph 1900, the median for the group administered a placebo is approximately 8 switches per trial, while the median for the group administered arbaclofen is approximately 10 switches per trial. Thus, the graph 1900 shows that arbaclofen increased the perceptual switch rate.

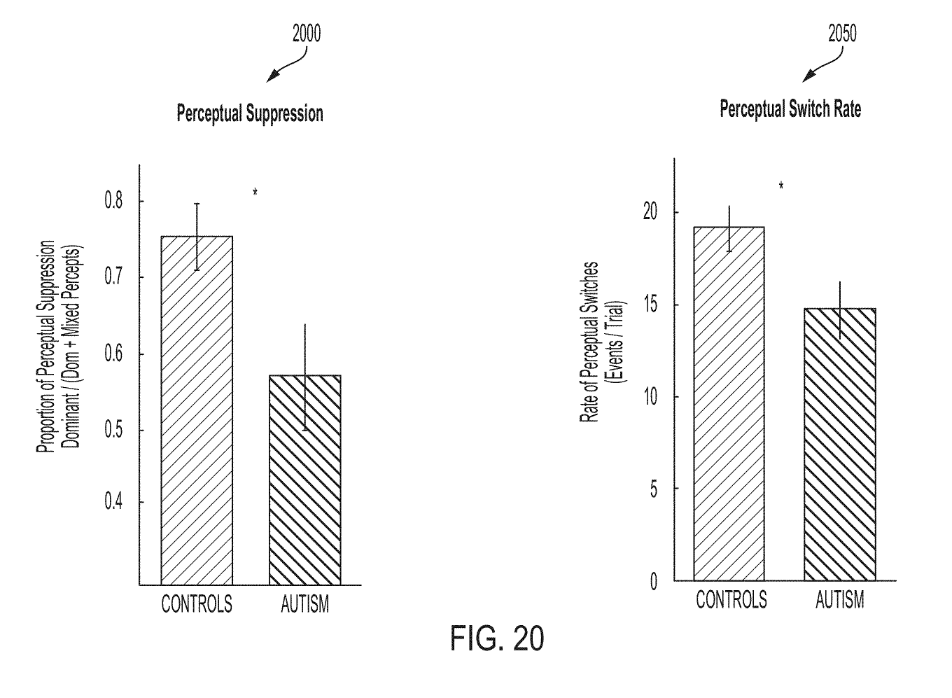

FIG. 20 shows bar graphs 2000, 2050 comparing the proportion of perceptual suppression (e.g., dominant/dominant+mixed percepts) in the control group and ASC group in the first graph with the number of switches per trial of the same groups in the second graph, according to some embodiments. Graph 2000 compares the proportion of perceptual suppression for the control and ASC groups, showing that the median for the control group is a proportion of perceptual suppression of approximately 0.75, while the median for the ASC group is a proportion of perceptual suppression of less than 0.6. Graph 2050 compares the number of switches per trial for the control and ASC groups, showing that the median for the control group is approximately 19 switches per trial, while the median for the ASC group is approximately 15 switches per trial. The post-study survey report responses corresponded to the effects shown in FIGS. 18-20, in which patients reported seeing separate images during the rivalry experiment of the second day, and mixed images during the rivalry experiment of the third day.

Techniques operating according to the principles described herein may be implemented in any suitable manner. Included in the discussion above are a series of flow charts showing the steps and acts of various processes that are used to record and analyze electrophysiological data (e.g., EEG data). The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application, such as geospatial assessment, web serving, and/or database management, and/or the like.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 210 of FIG. 3 (i.e., as a portion of the computing device 304) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 3, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

FIG. 3, as discussed above, illustrates one exemplary implementation of a computing device 304 that may be used in a system implementing techniques described herein, although others are possible. It should be appreciated that FIG. 3 is intended neither to be a depiction of necessary components for a computing device to operate as a voter platform in accordance with the principles described herein, nor a comprehensive depiction.

Computing device 304 may be, for example, a desktop or laptop personal computer, a personal digital assistant (PDA), a smart mobile phone, a server, a wireless access point or other networking element, or any other suitable computing device. Network adapter 308 may be any suitable hardware and/or software to enable the computing device 304 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 310 may be adapted to store data to be processed and/or instructions to be executed by processor 306. Processor 306 enables processing of data and execution of instructions. The data and instructions may be stored on the computer-readable storage media 310.

The data and instructions stored on computer-readable storage media 310 may comprise computer-executable instructions implementing techniques which operate according to the principles described herein. In the example of FIG. 3, computer-readable storage media 310 stores computer-executable instructions implementing various facilities and storing various information as described above.

While not illustrated in FIG. 3, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

1. A computerized method for analyzing brain activity of a subject associated with the subject's vision to identify signs associated with binocular rivalry, the method comprising:
receiving sensed electrical activity of a subject's brain over a time period while the subject is exposed to a visual stimulus, wherein the sensed electrical activity comprises:
a first frequency band associated with a first frequency of a first image presented to the subject's left eye; and
a second frequency band associated with a second frequency of a second image presented to the subject's right eye;
determining, based on the first and second frequency bands, a set of events in the time period, wherein an event in the set of events is associated with a change from a previous perceptual event triggered by observation of the first and second images by the subject to a new perceptual event triggered by observation of the first and second images by the subject;
determining, based on the set of events, a metric for the subject; and
analyzing the metric to determine whether the subject exhibits signs associated with a condition that is associated with binocular rivalry.

2. The computerized method of claim 1, wherein determining the metric comprises:
determining an average perceptual event rate;
determining a depth of perceptual suppression based on a ratio of dominant to mixed percepts; or both.

3. The computerized method of claim 1, wherein:
the first frequency band comprises a power of the first frequency band over the time period;
the second frequency band comprises a power of the second frequency band over the time period; and
determining the set of events comprises analyzing the data of the first frequency band and the second frequency band using a Fourier transform analysis to determine the average perceptual event rate for the subject.

4-7. (canceled)

8. A computerized apparatus for analyzing brain activity of a subject associated with the subject's vision to identify signs associated with binocular rivalry, the system comprising a processor in communication with a memory storing instructions that, when executed by the processor, cause the processor to:
receive sensed electrical activity of a subject's brain over a time period while the subject is exposed to a visual stimulus, wherein the sensed electrical activity comprises:
a first frequency band associated with a first frequency of a first image presented to the subject's left eye; and
a second frequency band associated with a second frequency of a second image presented to the subject's right eye;
determine, based on the first and second frequency bands, a set of events in the time period, wherein an event in the set of events is associated with a change from a previous perceptual event triggered by observation of the first and second images by the subject to a new perceptual event triggered by observation of the first and second images by the subject;
determine, based on the set of events, a metric for the subject; and
analyze the metric to determine whether the subject exhibits signs associated with a condition that is associated with binocular rivalry.

9. The computerized apparatus of claim 8, wherein determining the metric comprises:
determining an average perceptual event rate;
determining a depth of perceptual suppression based on a ratio of dominant to mixed percepts; or both.

10. The computerized apparatus of claim 8, wherein:
the first frequency band comprises a power of the first frequency band over the time period;
the second frequency band comprises a power of the second frequency band over the time period; and
determining the set of events comprises analyzing the data of the first frequency band and the second frequency band using a Fourier transform analysis to determine the average perceptual event rate for the subject.

11-14. (canceled)

15. A method of evaluating a subject comprising:
providing a visual stimulus to the subject; and
evaluating brain activity of the subject according to the method of claim 1 to determine a metric,
wherein the subject is likely to have a condition that is associated with binocular rivalry if the metric is less than a reference metric.

16. The method of claim 15, wherein the metric is average perceptual event rate.

17. The method of claim 15, wherein the metric is depth of perceptual suppression based on a ratio of dominant to mixed percepts; or both.

18. The method of claim 15, wherein the visual stimulus comprises:
presenting to the subject a first frequency-tagged image to the left eye and a second frequency-tagged image to the right eye, wherein the frequency of the first frequency-tagged image is different from the frequency of the second frequency-tagged image.

19-46. (canceled)

47. A method of determining the efficacy of a treatment in a subject suffering from a condition that is associated with binocular rivalry, the method comprising:
providing a visual stimulus to the subject; and
detecting brain activity of the subject by the method according to claim 1 to determine a metric;
wherein the treatment is said to be efficacious if the metric is increased after the administration of the treatment.

48. The method of claim 47, wherein the metric is average perceptual event rate.

49. The method of claim 47, wherein the metric is depth of perceptual suppression based on a ratio of dominant to mixed percepts; or both.

50. The method of claim 47, wherein the visual stimulus comprises:
presenting to the subject a first frequency-tagged image to the left eye and a second frequency-tagged image to the right eye, wherein the frequency of the first frequency-tagged image is different from the frequency of the second frequency-tagged image.

51-70. (canceled)

71. A method of treating a subject diagnosed with a condition that is associated with binocular rivalry, the method comprising:
providing a visual stimulus to the subject; and
detecting brain activity of the subject according to the method of claim 1 to determine a metric; and
administering a treatment to the subject if the metric of the subject is less than a reference metric.

72. A method of treating a subject diagnosed with a condition that is associated with binocular rivalry, the method comprising:
directing or ordering a test on the subject to determine a metric according to the method of claim 1; and
administering a treatment to the subject if the metric of the subject is less than a reference metric.

73. A method of treating a subject diagnosed with a condition that is associated with binocular rivalry, the method comprising:
selecting the subject on the basis that the subject has a metric that is less than a reference metric; and
administering a therapeutic treatment to the subject.

74-75. (canceled)

76. The method of claim 71, wherein the metric is average perceptual event rate.

77. The method of claim 71, wherein the metric is depth of perceptual suppression based on a ratio of dominant to mixed percepts; or both.

78. The method of claim 71, wherein the treatment comprises a behavioral treatment or administration of a pharmaceutical agent.

79-85. (canceled)
